(19) United States
(12) Patent Application Publication
(10) Pub. No.: US 2009/0227469 A1
(43) Pub. Date: Sep. 10, 2009

(54) CELLS AND ASSAYS FOR USE IN DETECTING LONG QT SYNDROME

(76) Inventors: Bruce R. Conklin, San Francisco, CA (US); Katriina Aalto-Setala, Tampere (FI)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/401,368

(22) Filed: Mar. 10, 2009

Related U.S. Application Data
(60) Provisional application No. 61/068,937, filed on Mar. 10, 2008.

(51) Int. Cl. C40B 30/06; C40B 40/02; C12Q 1/02

(52) U.S. Cl. 506/10; 506/14; 435/29

(57) ABSTRACT

The present disclosure provides induced pluripotent stem (iPS) cells, and induced multipotent stem (iMS) cells, and progeny thereof, which cells include a gene encoding a polypeptide that regulates the QT interval. The present disclosure further provides panels of cardiomyocytes suitable for use in screening compounds for an effect on the QT interval. The cells and panels of cells can be used in a variety of applications, which are also provided.

### CROSS-REFERENCE

This application claims the benefit of U.S. Provisional Patent Application No. 61/068,937, filed Mar. 10, 2008, which application is incorporated herein by reference in its entirety.

### BACKGROUND

The long QT syndrome (LQTS) is a heart condition associated with prolongation of repolarisation (recovery) following depolarisation (excitation) of the cardiac ventricles. Individuals with LQTS display a prolonged QT interval, as detected by electrocardiogram. The Q wave corresponds to the beginning of ventricular depolarization while the T wave corresponds to ventricular repolarization. The clinical features of LQTS result from episodic ventricular tachyarrhythmias, such as torsade de pointes and ventricular fibrillation. A prolonged QT interval can cause cardiac arrhythmias, a leading cause of death in the Western world.

The two most common types of LQTS are genetic and drug-induced. Acquired (drug-induced) LQTS is the single most common reason for drugs to be withdrawn from clinical trials, causing major setbacks to drug discovery efforts. Rare genetic forms of LQTS due to a mutation in the KCNH2 (HERG) potassium channel, or other genes, increase the risk for sudden death. Some long QT alleles are dominant, while others are recessive.

There is a need in the art for a test for drug-induced LQTS. Current tests for drug-induced LQTS include use of non-human animal models, or fibroblasts, that express human HERG ion channels at high levels. However, non-human animal models (such as rabbits and dogs) have different cardiac physiology than humans, and fibroblasts expressing cardia ion channels lack the regulatory network found in human myocytes. Thus, the non-human animal models and the fibroblasts currently in use do not reflect human cardiomyocyte physiology.

### LITERATURE

Chiang and Roden (2000) J. Am. Coll. Cardiol. 36:1; U.S. Pat. No. 7,179,597; U.S. Patent Publication No. 2004/0106095

### SUMMARY OF THE INVENTION

The present disclosure provides induced pluripotent stem (iPS) cells, and induced multipotent stem (iMS) cells, and progeny thereof, which cells include a gene encoding a polypeptide that regulates the QT interval. The present disclosure further provides panels of cardiomyocytes suitable for use in screening compounds for an effect on the QT interval. The cells and panels of cells can be used in a variety of applications, which are also provided.

### BRIEF DESCRIPTION OF THE DRAWINGS

FIGS. 1A and 1B depict ion channels in cardiac conduction.

FIGS. 2A and 2B depict amino acid sequences of KVLQT1. FIG. 2A depicts a wild-type sequence; SEQ ID NO:1); FIG. 2B depicts a mutant sequence; SEQ ID NO:2).

FIG. 3 depicts an amino acid sequence of HERG (SEQ ID NO:3).

FIG. 4 depicts an amino acid sequence of SCN5A (SEQ ID NO:4).

FIGS. 5A-D depict ANK2 amino acid sequences. FIGS. 5A-C depict an amino acid sequence of ANK2 isoform 1 (SEQ ID NO:5). FIG. 5D depicts an amino acid sequence of ANK2 isoform 2 (SEQ ID NO:6).

FIG. 6 depicts an amino acid sequence of MinK (SEQ ID NO:7).

FIG. 7 depicts an amino acid sequence of MiRP1 (SEQ ID NO:8).

FIG. 8 depicts an amino acid sequence of Kir2.1 (SEQ ID NO:9).

FIGS. 9A and 9B depict an exemplary assay for detection of possible LQTS-inducing activity.

### DEFINITIONS

The term “long QT syndrome,” or “LQTS,” refers any disease or disorder, whether congenital or acquired, that is caused by defects in the ion channel mechanism controlling cardiac cell excitation.

A “gene involved in long QT syndrome (LQTS)” (e.g., an LQTS-associated gene) refers to any gene that contributes (by deletion, mutation, variation in activity) to a defect in the ion channel mechanism controlling cardiac cell excitation. A gene involved in LQTS may be, for example, an ion channel gene, or other gene that is involved in the ion channel regulation and thus controls cardiac cell excitation, such as scaffolding and cytoskeletal proteins, a phosphatase or a kinase. A “cardiac ion channel gene involved in LQTS” refers to any gene encoding a cardiac ion channel that contributes (by deletion, mutation, variation in activity) to a defect in the ion channel mechanism controlling cardiac cell excitation. The term therefore includes both wild-type and mutant cardiac ion channel genes. Gene products (e.g., polypeptides) encoded by the genes control, directly or indirectly, the QT interval.

The term “induced pluripotent stem cell” (or “iPS cell”), as used herein, refers to a pluripotent stem cell induced from a somatic cell, e.g., a differentiated somatic cell. iPS cells are capable of self-renewal and differentiation into cell fate-committed stem cells, including neural stem cells, as well as various types of mature cells. Non-limiting examples of such mature cells include cells of mesodermal lineage and cardiomyocytes. iPS may also be capable of differentiation into cardiac progenitor cells.

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene may differ from each other in a single nucleotide, or several nucleotides, and may include substitutions, deletions, and insertions of nucleotides. An allele of a gene may also be a form of a gene containing a mutation.

As used herein, an “annotation” is a comment, explanation, note, link, or metadata about a data element. Annotations may include pointers to external objects or external data. An annotation may optionally include information about an author who created or modified the annotation, as well as information about when that creation or modification occurred. In one embodiment, a memory comprising a plurality of data structures organized by annotation category provides a database through which information from multiple databases, public or private, may be accessed, assembled, and processed.

A “data element” represents a property of a cell in a subject cell panel, which can include: 1) information regarding a mutation in an LQTS-associated gene; 2) information regarding the individual from whom the cardiomyocyte originated; and 3) information regarding the effect, if any, of a given drug on the function of an ion channel in a cardiomyocyte in a subject cell panel. A data element can be represented for example, by an alphanumeric string (e.g., representing bases), by a number, by “plus” and “minus” symbols or other symbols, by a color hue, by a word, or by another form (descriptive or nondescriptive) suitable for computation, analysis and/or processing for example, by a computer or other machine or system capable of data integration and analysis.

As used herein an “annotation category” is a human readable string to annotate the logical type the object comprising its plurality of data elements represents. Data structures that contain the same types and instances of data elements may be assigned identical annotations, while data structures that contain different types and instances of data elements may be assigned different annotations.

As used herein, a “cell identifier” or an “identifier corresponding to a cell” refers to a string of one or more characters (e.g., alphanumeric characters), symbols, images or other graphical representation(s) associated with a cell in a subject cell panel, where the cell can comprise a mutation in an LQTS-associated gene such that the identifier provides a “shorthand” designation for the mutation. An identifier may comprise descriptive information.

A “computer-based system” refers to the hardware means, software means, and data storage means used to analyze the information generated with a subject cell panel. The minimum hardware of the computer-based systems of the present disclosure comprises a central processing unit (CPU), input means, output means, and data storage means. A skilled artisan can readily appreciate that many computer-based systems are available which are suitable for use in connection with annotating a subject cell panel. The data storage means may comprise any manufacture comprising a recording of the present information as described above, or a memory access means that can access such a manufacture.

A “processor” references any hardware and/or software combination which will perform the functions required of it. For example, a processor may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, UBS, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cardiomyocyte” includes a plurality of such cardiomyocytes and reference to “the drug” includes reference to one or more drug and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides induced pluripotent stem (iPS) cells, and induced multipotent stem (iMS) cells, and progeny thereof, which cells include a gene encoding a polypeptide that regulates the QT interval. The present disclosure further provides panels of cardiomyocytes suitable for use in screening compounds for an effect on the QT interval. The cells and panels of cells can be used in a variety of applications, which are also provided. For example, the present disclosure further provides assays for determining whether a given compound has the potential to induce LQTS in an individual. The present disclosure also provides assays for identifying an agent that reduces the QT interval and/or ameliorates LQTS.

As noted above, current tests for drug-induced LQTS include use of non-human animal models, or fibroblasts, that express human HERG ion channels at high levels. However, non-human animal models (such as rabbits and dogs) have different cardiac physiology than humans, and fibroblasts expressing cardiac ion channels lack the regulatory network found in human myocytes. Thus, the non-human animal models and the fibroblasts currently in use do not reflect human cardiomyocyte physiology.

Induced pluripotent stem (iPS) cells or induced multipotent cells (iMS) can be generated from a somatic cell from an individual, and the iPS or iMS cell can be induced to differentiate in vitro into a cardiomyocyte or a cardiomyocyte progenitor. In some embodiments, any pluripotent cell is induced to differentiate into a cardiomyocyte or cardiomyocte progenitor. In some embodiments, any pluripotent cell other than an embryonic stem (ES) cell is induced to differentiate into a cardiomyocyte or cardiomyocyte progenitor.

Use of such cells to identify compounds that may induce LQTS is advantageous, because, unlike fibroblasts currently in use, such cells are cardiomyocytes, and thus would more closely reflect the in vivo effect of a drug that induces LQTS. Furthermore, the amount of drug used in a test involving cardiomyocytes generated from iPS or iMS is more readily correlated with the dose that would be used in vivo.

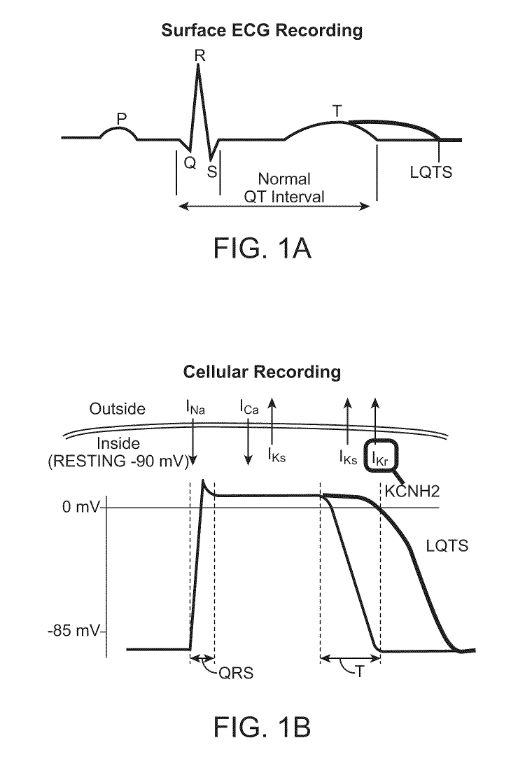

FIGS. 1A and 1B depict ion channels in cardiac conduction. Cardiac electrical conduction involves several ion currents. FIG. 1A depicts a surface electrocardiogram (ECG), showing the QT interval. The QT interval is a measure of the time between the start of the Q wave and the end of the T wave in the heart's electrical cycle. The QTc is a correction for the QT interval at different heart rates. FIG. 1B depicts electrial potential of individual cardiac myocytes. Many ion channels control rhythmic cardiac contraction: I_{Nz}, I_{Ca}, I_{Ks}, and I_{Kr}. The I_{Kr} (HERG; KCNH2) is most often involved with drug-induced LQTS. Genetic mutations in this channel (KCNH2) can result in vulnerability to drug-induced LQTS.

Thus, among the possible uses of a cardiomyocyte generated from, e.g., an iPS or an iMS cell obtained from an individual, is its use in predictive cardiotoxicology, e.g., determining whether a test compound has the potential to induce LQTS in an individual. A cardiomyocyte generated from, e.g., an iPS or an iMS cell obtained from an individual, can also be used in the area of personalized medicine, e.g., assessing whether a given compound is likely to induce LQTS in an individual, and, based on that assessment, making a recommendation as to whether the compound should be administered to the individual, or whether an alternative compound should be administered.

### Induced Pluripotent and Induced Multipotent Stem Cells

The present disclosure provides induced pluripotent stem (iPS) cells, induced multipotent stem cells (iMS), and progeny thereof, that include a gene encoding a polypeptide that controls the QT interval. The gene encoding a polypeptide that controls the QT interval will in some embodiments be wild-type, e.g., will encode a functional polypeptide, e.g., a functional ion channel. In other embodiments, the iPS cell or iMS cell will include a mutant gene encoding a mutant polypeptide, where the mutant polypeptide contributes to one or more symptoms associated with LQTS.

For example, where the iPS cell or iMS cell comprises a mutant KCNQ1, the amount of repolarizing current that is required to terminate the action potential is reduced, leading to an increase in the action potential duration (APD). As another example, where the iPS cell or iMS cell comprises a mutant HERG, a reduction in repolarizing current is observed. As another example, where the iPS cell or iMS cell comprises a mutant SCN5A, depolarizing current through the channel late in the action potential is thought to prolong APD.

iPS cells or iMS cells are generated from mammalian cells (including mammalian somatic cells) using, e.g., known methods. Examples of suitable mammalian cells include, but are not limited to: fibroblasts, skin fibroblasts, dermal fibroblasts, bone marrow-derived mononuclear cells, skeletal muscle cells, adipose cells, peripheral blood mononuclear cells, macrophages, hepatocytes, keratinocytes, oral keratinocytes, hair follicle dermal cells, epithelial cells, gastric epithelial cells, lung epithelial cells, synovial cells, kidney cells, skin epithelial cells, pancreatic beta cells, and osteoblasts.

Mammalian cells used to generate iPS cells or iMS cells can originate from a variety of types of tissue including but not limited to: bone marrow, skin (e.g., dermis, epidermis), muscle, adipose tissue, peripheral blood, foreskin, skeletal muscle, and smooth muscle. The cells used to generate iPS cells or iMS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPS cells or iMS cells can be derived from tissue of a non-embryonic subject, a neonatal infant, a child, or an adult. In some embodiments, cells used to generate iPS cells or iMS cells are obtained from a post-natal human. Cells used to generate iPS cells or iMS cells can be derived from neonatal or post-natal tissue collected from a subject within the period from birth, including cesarean birth, to death. For example, the tissue source of cells used to generate iPS cells or iMS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

In general, cells used to generate an iPS cells or iMS cells are substantially genetically identical to a somatic cell from a post-natal human, e.g., are substantially genetically identical to a somatic cell of the post-natal human from which the cell used to generate the iPS cell or the iMS cell is derived.

iPS cells or iMS cells produce and express on their cell surface one or more of the following cell surface antigens: SSEA-3, SSEA-4, TRA-1-60, TRA-1-81, TRA-2-49/6E (alkaline phophatase), and Nanog. In some embodiments, iPS cells produce and express on their cell surface SSEA-3, SSEA-4, TRA-1-60, TRA-1-81, TRA-2-49/6E, and Nanog. iPS cells or iMS cells express one or more of the following genes: Oct-3/4, Sox2, Nanog, GDF3, REX1, FGF4, ESG1, DPPA2, DPPA4, and hTERT. In some embodiments, an iPS cell or an iMS cell expresses Oct-3/4, Sox2, Nanog, GDF3, REX1, FGF4, ESG1, DPPA2, DPPA4, and hTERT.

### Sources of Cells

As noted above, an iPS cell or an iMS cell used to generate a cardiomyocyte can be derived from a variety of cells of an individual (e.g., a post-natal human). The source of cells used to generate an iPS cell or an iMS cell can include: 1) an individual who has no known mutation in any polypeptide associated with control of the QT interval and who has not experienced LQTS (has not had an LQTS episode); 2) an individual who has a mutation in polypeptide associated with control of the QT interval, where the mutation is known to be associated with increased risk of LQTS; 3) an individual who has no known mutation in any polypeptide associated with control of the QT interval and who has an LQTS episode or symptom at least once. LQTS symptoms include, e.g., fainting, lightheadedness, blurred vision, heart palpitations, irregular heartbeat, and seizure.

As noted above, the tissue source of cells used to generate iPS cells or iMS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

The source of cells used to generate an iPS cell can include 1) individuals of a particular genetic background, e.g., individuals of a particular major histocompatibility complex (MHC) (or human leukocyte antigen; HLA) haplotype, etc.; 2) individuals of a particular race, e.g., Caucasian individuals; African individuals or individuals of African descent; Asian individuals or individuals of Asian descent; native American individuals; African Americans; Hispanic/Latino individuals; etc.; 3) female individuals; 4) male individuals; 5) individuals having a particular disease state, e.g., individuals with chronic kidney disease such as end-stage renal failure; individuals with known cardiovascular disease; individuals with liver disease; etc.; 6) individuals known to have been exposed chronically to an environmental toxin; 7) individuals who are habitual smokers of tobacco products (e.g., cigarettes); 8) individuals who are considered to be heavy consumers of alcohol; 9) individuals of a particular national or geographic origin; and the like.

In some embodiments, the individual is of a particular HLA haplotype. HLA antigens are the products of multiple, closely linked genes on a single chromosome usually inherited as an intact unit. The HLA gene complex contains at least four loci known as HLA-A, -B, -Cw, and -DR. There are two classes of HLA antigens. Class I is comprised of HLA-A, -B, and -Cw. Class II is comprised of HLA-DR and -DQ. An individual has two of each A, B, Cw, and DR alleles, where one set of A, B, Cw, and DR (a “haplotype”) is inherited from each parent. Methods of determining an individual's HLA haplotype are known in the art; see, e.g., U.S. Pat. No. 7,030,292 and references cited therein. Information regarding HLA haplotypes for various populations is publicly available. See, e.g., Mori et al. (1997) Transplantation 64(7):1017; and on the internet at hapmap.org.

### Generating iPS Cells and iMS Cells

Methods of generating iPS cells are known in the art, and a wide range of methods can be used to generate iPS cells. See, e.g., Takahashi and Yamanaka (2006) Cell 126:663-676; Yamanaka et al. (2007) Nature 448:313-7; Wernig et al. (2007) Nature 448:318-24; Maherali (2007) Cell Stem Cell 1:55-70; Maherali and Hochedlinger (2008) Cell Stem Cell 3:595-605; Park et al. (2008) Cell 134:1-10; Dimos et al. (2008) Science 321:1218-1221; Blelloch et al. (2007) Cell Stem Cell 1:245-247; Stadtfeld et al. (2008) Science 322:945-949; Stadtfeld et al. (2008) 2:230-240; Okita et al. (2008) Science 322:949-953; Woltgen, et al. (2009) Nature doi:10.1038/nature 07863, published online Mar. 1, 2009, and Kaji et al. (2009) Nature doi:10.1038/nature07864, published online Mar. 1, 2009.

In some embodiments, iPS cells or iMS cells are generated from somatic cells by forcing expression of a set of factors in order to promote increased potency of a cell or de-differentiation. Forcing expression can include introducing expression vectors encoding polypeptides of interest into cells, introducing exogenous purified polypeptides of interest into cells, or contacting cells with a reagent that induces expression of an endogenous gene encoding a polypeptide of interest.

Forcing expression may include introducing expression vectors into somatic cells via use of moloney-based retroviruses (e.g., MLV), lentiviruses (e.g., HIV), adenoviruses, protein transduction, transient transfection, or protein transduction. In some embodiments, the moloney-based retroviruses or HIV-based lentiviruses are pseudotyped with envelope from another virus, e.g. vesicular stomatitis virus g (VSV-g) using known methods in the art. See, e.g. Dimos et al. (2008) Science 321:1218-1221.

In some embodiments, iPS cells or iMS cells are generated from somatic cells by forcing expression of Oct-3/4 and Sox2 polypeptides. In some embodiments, iPS cells or iMS cells are generated from somatic cells by forcing expression of Oct-3/4, Sox2 and Klf4 polypeptides. In some embodiments, iPS cells are generated from somatic cells by forcing expression of Oct-3/4, Sox2, Klf4 and c-Myc polypeptides. In some embodiments, iPS cells or iMS cells are generated from somatic cells by forcing expression of Oct-4, Sox2, Nanog, and LIN28 polypeptides.

For example, iPS cells or iMS cells can be generated from somatic cells by genetically modifying the somatic cells with one or more expression constructs encoding Oct-3/4 and Sox2. As another example, iPS cells or iMS cells can be generated from somatic cells by genetically modifying the somatic cells with one or more expression constructs comprising nucleotide sequences encoding Oct-3/4, Sox2, c-myc, and Klf4. As another example, iPS cells or iMS cells can be generated from somatic cells by genetically modifying the somatic cells with one or more expression constructs comprising nucleotide sequences encoding Oct-3/4, Sox2, Klf4, and c-Myc. As another example, iPS cells can be generated from somatic cells by genetically modifying the somatic cells with one or more expression constructs comprising nucleotide sequences encoding Oct-4, Sox2, Nanog, and Lin28.

In some embodiments, cells undergoing induction of pluripotency as described above, to generate iPS cells or iMS cells, are contacted with additional factors which can be added to the culture system, e.g., included as additives in the culture medium. Examples of such additional factors include, but are not limited to: histone deacetylase (HDAC) inhibitors, see, e.g. Huangfu et al. (2008) Nature Biotechnol. 26:795-797; Huangfu et al. (2008) Nature Biotechnol. 26: 1269-1275; DNA demethylating agents, see, e.g., Mikkelson et al (2008) Nature 454, 49-55; histone methyltransferase inhibitors, see, e.g., Shi et al. (2008) Cell Stem Cell 2:525-528; L-type calcium channel agonists, see, e.g., Shi et al. (2008) 3:568-574; Wnt3a, see, e.g., Marson et al. (2008) Cell 134:521-533; and siRNA, see, e.g., Zhao et al. (2008) Cell Stem Cell 3: 475-479.

In some embodiments, iPS cells or iMS cells are generated from somatic cells by forcing expression of Oct3/4, Sox2 and contacting the cells with an HDAC inhibitor, e.g., valproic acid. See, e.g., Huangfu et al. (2008) Nature Biotechnol. 26: 1269-1275. In some embodiments, iPS cells or iMS cells are generated from somatic cells by forcing expression of Oct3/4, Sox2, and Klf4 and contacting the cells with an HDAC inhibitor, e.g., valproic acid. See, e.g., Huangfu et al. (2008) Nature Biotechnol. 26:795-797.

### Inducing an iPS Cell or an iMS Cell to Undergo Cardiomyogenesis

An iPS cell or an iMS cell can be induced to undergo cardiomyogenesis using any of a variety of known methods. For example, an iPS or an iMS cell can be co-cultured with visceral endoderm-like cells (see, e.g., Mummery et al. (2003) Circulation 107:2733). An iPS cell or an iMS cell can also be induced to undergo cardiomyogenesis without co-culture with a feeder cell or other cell. For example, as described in U.S. Pat. No. 7,297,539, cells can be cultured in Matrigel™-coated plastic plates or wells in the presence of conditioned medium, where the conditioned medium is generated by irradiated primary fibroblasts. Differentiation is facilitated by nucleotide analogs that affect DNA methylation (such as 5-aza-deoxy-cytidine), growth factors, and bone morphogenic proteins. The cells can be further enriched by density-based cell separation, and maintained in media containing creatine, camitine, and taurine (see, e.g., WO 2003/006950); and/or prostaglandin alone or in combination with other factors including essential minerals such as transferrin and selenium, small molecules selected from the group including a p38 MAPK inhibitor such as SB203580 and protein growth factors of the FGF, IGF and BMP families such as but not limited to IGF1, FGF2, BMP2, BMP4 and BMP6, and insulin.

Whether an iPS cell or iMS cell or progenitor cell has differentiated into a cardiomyocyte can be readily determined. For example, in some embodiments, differentiation into a cardiomyocyte is ascertained by detecting cardiomyocyte-specific markers produced by the cell. For example, the cardiomyocytes express cardiac transcription factors, sarcomere proteins, and gap junction proteins. Suitable cardiomyocyte-specific proteins include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor.

Whether an iPS cell or iMS cell or progenitor cell has differentiated into a cardiomyocyte can also be determined by detecting responsiveness to pharmacological agents such as β-adrenergic agonists (e.g., isoprenaline), adrenergic β-antagonists (e.g., esmolol), cholinergic agonists (e.g., carbochol), and the like.

Whether an iPS cell or iMS cell or progenitor cell has differentiated into a cardiomyocyte can also be determined by detecting electrical activity of the cells. Electrical activity can be measured by various methods, including extracellular recording, intracellular recording (e.g., patch clamping), and use of voltage-sensitive dyes. Such methods are well known to those skilled in the art.

As discussed above, in some embodiments, a cardiomyocyte is generated from an iPS cell or an iMS cell. In addition to iPS cells and iMS cells, any pluripotent, multipotent, or other non-lineage-committed cell can be used to generate a cardiomyocyte, e.g., for inclusion in a subject cell panel and/or use in a subject method. In some embodiments, the cell used to generate a cardiomyocyte is any multipotent or pluripotent cell other than an embryonic stem (ES) cell.

Polypeptides Associated with Control of QT Interval

In some embodiments, a gene involved in LQTS is a human cardiac ion channel gene or a gene that encodes a polypeptide that regulates a cardiac ion channel. Human cardiac ion channel genes involved in control of the QT interval include, but are not limited to, the following, where the gene is provided, followed by the encoded protein and the associated type of LQTS in parentheses: KCNQ1 (encoding KvLQT1; mutations in KvLQT1 are associated with LQT1 and affect potassium channel function (I_{Ks})); KCNH2 (encoding HERG; mutations in HERG are associated with LQT2 and affect potassium channel function (I_{Kr})); SCN5A (encoding Nav1.5; mutations in Nav1.5 are associated with LQT3 and affect sodium channel function (I_{Na})); ANK2 (encoding ankyrin-B; mutations in ankyrin-B are associated with LQT4, and affect sodium, potassium, and calcium channel function); KCNE1 (encoding MinK; mutations in MinK are associated with LQT5 and affect potassium channel function (I_{Ks})); KCNE2 (encoding MiRP1; mutations in MiRP1 are associated with LQT6 and affect potassium channel function (I_{Kr})); KCNJ2 (encoding Kir2.1; mutations in Kir2.1 are associated with LQT7 and affect potassium channel function (I_{Kl})); CACNA1c (encoding Cav1.2; mutations in Cav1.2 are associated with LQT8 and affect calcium channel function (I_{Ca-Lalpha})); CAV3 (encoding caveolin-3; mutations in caveolin-3 are associated with LQT9 and affect sodium channel function (I_{Na})); and SCN4B (encoding Navβ4; mutations in Navβ4 are associated with LQT10 and affect sodium channel function (I_{Na})).

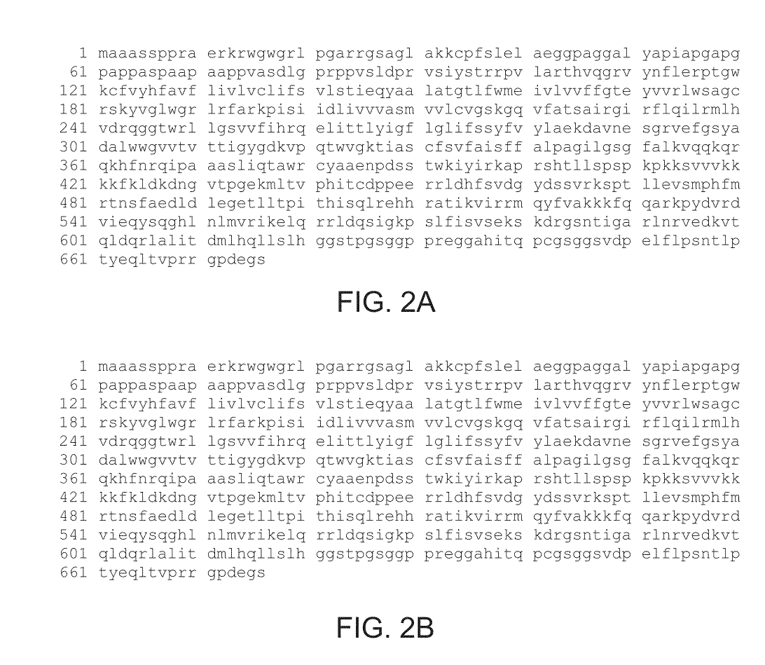

KVLQT1 (encoded by the KCNQ1 gene) is a voltage-gated potassium channel. A wild-type amino acid sequence of human KVLQT1 is found under GenBank Accession No. AAC51776, and is depicted in FIG. 2A. A nucleotide sequence encoding the amino acid sequence set forth in AAC51776 is found under GenBank Accession No. AF000571.

Mutations in KCNQ1 are associated with LQT1. Mutations in KCNQ1 can cause LQTS by reducing the amount of repolarizing current that is required to terminate the action potential, leading to an increase in the action potential duration (APD). Mutations can cause decreased outward potassium current. Mutations that map to the KvLQT1 gene on human chromosome 11 account for more than 50% of inherited LQTS. Mutant KVLQT1 polypeptides are known in the art, and the amino acid sequences of several mutant KVLQT1 polypeptides are known. See, e.g., the amino acid sequence set forth in GenBank Accession No. NP_{—}000209, and depicted in FIG. 2B; Neyroud et al. (1997) Nat. Genet. 15 (2), 186-189; Russell et al. (1996) Hum. Mol. Genet. 5 (9), 1319-1324; Wang et al. (1996) Nat. Genet. 12 (1), 17-23; Splawski et al. (2000) Circulation 102 (10), 1178-1185; Tanaka et al. (1997) Circulation 95: 565-7; U.S. Pat. No. 6,342,357; U.S. Pat. No. 6,277,978; and International Patent Publication No. WO 97/023598. For example, Shalaby et al. (1997) Circulation 96:1733-1736 found that substitutions of alanine with proline in the S2-S3 cytoplasmic loop (A177P) or threonine with isoleucine in the highly conserved signature sequence of the pore (T311I) yield inactive channels when expressed individually, whereas substitution of leucine with phenylalanine in the S5 transmembrane domain (L272F) yields a functional channel with reduced macroscopic conductance; however, all these mutants were found to inhibit wild-type KvLQT1 currents in a dominant-negative fashion. Missense mutations R243c, W248R, and E261K were associated with LQTS (Franqueza et al. (1999) J. Biol. Chem. Vol. 274, Issue 30, 21063-21070). Additional mutations in KCNQ1 that are associated with LQTS are depicted in Table 2 of U.S. Pat. No. 6,787,309. See, e.g., the exemplary mutations depicted in Table 1, below, which include the LQTS-associated KCNQ1 mutations from Table 2 of U.S. Pat. No. 6,787,309.

TABLE 1

| Nucleotide change | Amino acid change |
|---|---|
| del211-219 | del 71-73 |
| A332G | Y111C |
| del451-452 | A150fs/132 |
| T470G | F157C |
| G477 + 1A | M159sp |
| G477 + 5A | M159sp |
| G478A | E160K |
| del500-502 | F167W/del G168 |
| G502A | G168R |
| C520T | R174C |
| G521A | R174H |
| G532A | A178T |
| G532C | A178P |
| G535A | G179S |
| A551C | Y184S |
| G565A | G189R |
| insG567-568 | G189fs/94 |
| G569A | R190Q |
| del572-576 | L191fs/90 |
| G580C | A194P |
| C674T | S225L |
| G724A | D242N |
| C727T | R243C |
| G728A | R243H |
| T742C | W248R |
| T749A | L250H |
| G760A | V254M |
| G781A | E261K |
| T797C | L266P |
| G805A | G269S |
| G806A | G269D |
| C817T | L273F |
| A842G | Y281C |
| G898A | A300T |
| G914C | W305S |
| G961A | G306R |
| del921 − (921 + 2) | V307sp |
| G921 + 1T | V307sp |
| A922 − 1C | V307sp |
| G922 − 1C | V307sp |
| C926G | T309R |
| G928A | V310I |
| C932T | T311I |
| C935T | T312I |
| C939G | I313M |
| G940A | G314S |
| A944C | Y315S |
| A944G | Y315C |
| G949A | D317N |
| G954C | K318N |
| C958G | P320A |
| G973A | G325R |
| del1017-1019 | delF340 |
| C1022T | A341V |
| C1024T | L342F |
| C1031T | A344V |
| G1032A | A344sp |
| G1032C | A344sp |
| G1033C | G345R |
| G1034A | G345E |
| C1046G | S349W |
| T1058C | L353P |
| C1066T | Q356X |
| C1096T | R366W |
| G1097A | R366Q |
| G1097C | R366P |
| G1111A | A371T |
| T1117C | S373P |
| C1172T | T391I |
| T1174C | W392R |
| C1343G | P448R |
| C1522T | R518X |
| G1573A | A525T |
| C1588T | Q530X |
| C1615T | R539W |
| del6/ins7 | E543fs/107 |
| C1663T | R555C |
| C1697T | S566F |
| C1747T | R583C |
| C1760T | T587M |
| G1772A | R591H |
| G1781A | R594Q |
| del1892-1911 | P630fs/13 |
| insC1893-1894 | P631fs/19 |

In Tables 1-4, “del” indicates a deletion; “fs” indicates a frameshift; and “sp” indicates a splice mutation; “ins” indicates an insertion; “dupl” indicates a duplication; and “X” indicates a translation stop.

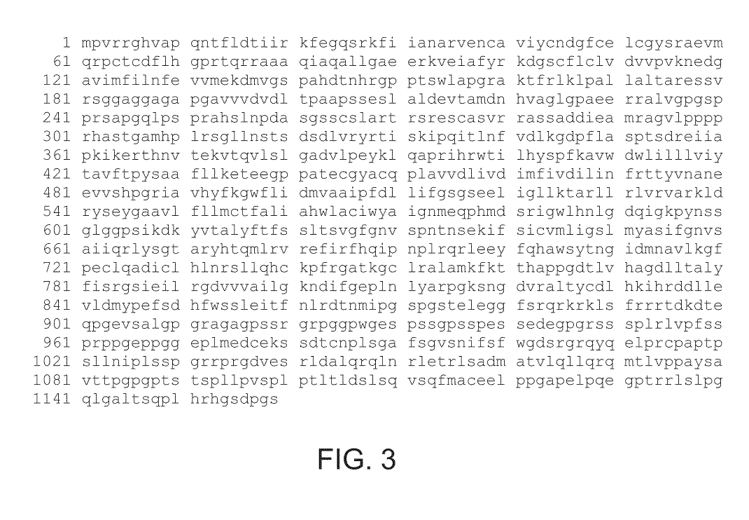

HERG (encoded by the KCNH2 gene) is a potassium channel. A wild-type amino acid sequence of human HERG is found under GenBank Accession No. AAA62473, and is depicted in FIG. 3. A nucleotide sequence encoding the amino acid sequence set forth in AAA62473 (and FIG. 3) is found under GenBank Accession No. U04270. A wild-type HERG amino acid sequence is provided as SEQ ID NO:2 in U.S. Pat. No. 7,297,489; the nucleotide sequence encoding SEQ ID NO:2 of U.S. Pat. No. 7,297,489 is presented as SEQ ID NO:1 in U.S. Pat. No. 7,297,489.

Mutations in HERG are associated with LQT2. Mutations in HERG can cause rapid closure of the potassium channels and decrease the normal rise in I_{Kr}. Mutant HERG polypeptides are known in the art, and the amino acid sequences of several mutant HERG polypeptides are known. See, e.g., Gong et al. (2007) Circulation 116 (1), 17-24; Curran et al. (1995) Cell 80 (5), 795-803; Newton-Cheh et al. (2007) Circulation 116 (10), 1128-1136; Tanaka et al. (1997) Circulation 95: 565-7; U.S. Pat. No. 7,297,489; Chen et al. (1999) J. Biol. Chem. Vol. 274, Issue 15, 10113-10118. Mutations include, e.g., F29L, N33T, G53R, R56Q, C66G, H70R, A78P, and L86R. Crotti et al. ((2005) Circulation 112:1251) reported that co-expression of K897T and A1116V mutations in KCNH1 resulted in significantly reduced current amplitude. Yoshida et al. ((2001) Am. J. Med. Genet. 98:348) reported a missense mutation (A490T) in the S2-S3 inner loop leading to LQTS. Additional HERG mutations associated with LQTS are depicted in Table 3 of U.S. Pat. No. 6,787,309; and in Table 7 of U.S. Pat. No. 7,297,489. Table 3 of U.S. Pat. No. 6,787,309 is reproduced below as Table 2.

TABLE 2

| Nucleotide change | Amino acid change |
|---|---|
| C87A | F29L |
| A98C | N33T |
| C132A | C44X |
| G140T | G47V |
| G157C | G53R |
| G167A | R56Q |
| T196G | C66G |
| A209G | H70R |
| C215A | P72Q |
| del221-251 | R73sf/31 |
| G232C | A78P |
| dup1234-250 | A83fs/37 |
| C241T | Q81X |
| T257G | L86R |
| insC422-423 | P141sf/2 |
| insC453-454 | P151fs/179 |
| dupl558-600 | L200sf/179 |
| insC724-725 | P241fs/89 |
| del885 | V295fs/63 |
| C934T | R312C |
| C1039T | P347S |
| G1128A | Q376sp |
| A1129-2G | Q376sp |
| del1261 | Y420fs/12 |
| C1283A | S428X |
| C1307T | T436M |
| A1408G | N470D |
| C1421T | T4741 |
| C1479G | Y493X |
| del1498-1524 | del500-508 |
| G1592A | R531Q |
| C1600T | R534C |
| T1655C | L552S |
| delT1671 | T556fs/7 |
| G1672C | A558P |
| G1681A | A561T |
| C1682T | A561V |
| G1714C | G572R |
| G1714T | G572C |
| C1744T | R582C |
| G1750A | G584S |
| G1755T | W585C |
| A1762G | N588D |
| T1778C | 1593T |
| T1778G | 1593R |
| G1801A | G601S |
| G1801A | G604S |
| G1825A | D609N |
| T1831C | Y611H |
| T1833 (A or G) | Y611X |
| G1834T | V612L |
| C1838T | T613M |
| C1841T | A614V |
| C1843G | L615V |
| G1876A | G626S |
| C1881G | F627L |
| G1882A | G628S |
| A1885G | N629D |
| A1886G | N629S |
| C1887A | N629K |
| G1888C | V630L |
| T1889C | V630A |
| C1894T | P632S |
| A1898G | N633S |
| A1912G | K638E |
| del1913-1915 | delK638 |
| C1920A | F640L |
| A1933T | M645L |
| del1951-1952 | L650fs/2 |
| G2044T | E682X |
| C2173 | Q725X |
| insT2218-2219 | H739fs/63 |
| C2254T | R752W |
| dup12356-2386 | V796fs/22 |
| del2395 | I798fs/10 |
| G2398 + 1C | L799sp |
| T2414C | F805S |
| T2414G | F805C |
| C2453T | S818L |
| G2464A | V822M |
| C2467T | R823W |
| A2582T | N861I |
| G2592 + 1A | D864sp |
| del2660 | K886fs/85 |
| C2750T | P917L |
| del2762 | R920fs/51 |
| C2764T | R922W |
| insG2775-2776 | G925fs/13 |
| del2906 | P968fs/4 |
| del2959-2960 | P986fs/130 |
| C3040T | R1014X |
| del3094 | G1031fs/24 |
| insG3107-3108 | G1036fs/82 |
| insC3303-3304 | P1101fs |

The SCN5A gene (encoding Nav1.5) encodes a sodium channel protein type 5 subunit alpha. The SCN5A (or Nav1.5) protein is an integral membrane protein and tetrodotoxin-resistant voltage-gated sodium channel subunit. Defects in the SCN5A gene are a cause of long QT syndrome type 3 (LQT3), an autosomal dominant cardiac disease. SCN5A encodes the cardiac sodium channel that is responsible for I_{Na}, the sodium current in the heart. LQTS-associated mutations in SCN5A can cause a gain-of-function. Bennett et al. (1995) Nature, Aug. 24, 1995; 376:683-685; Dumaine et al. (1999) Circ. Res., 85:803-809.

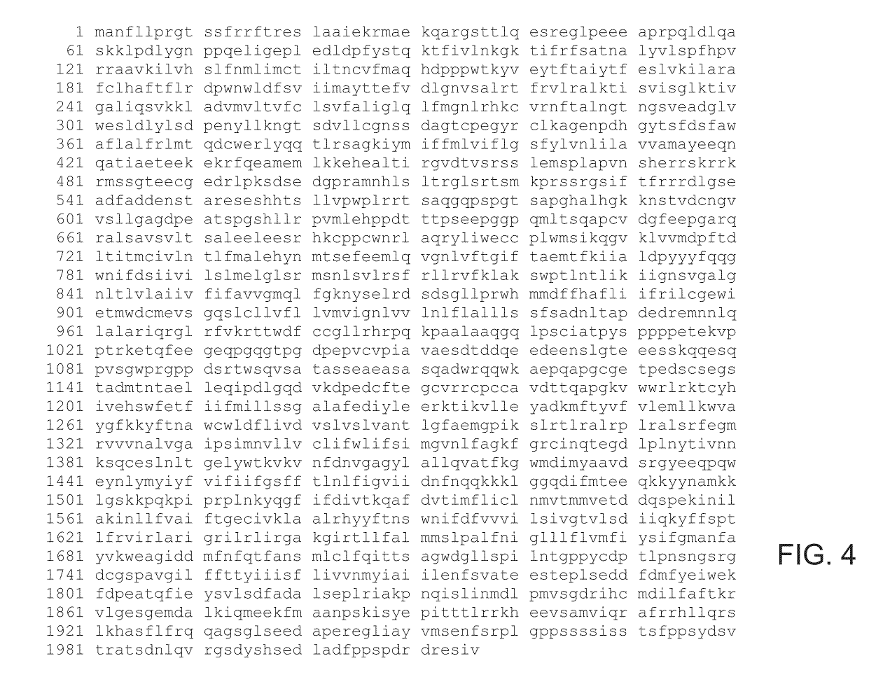

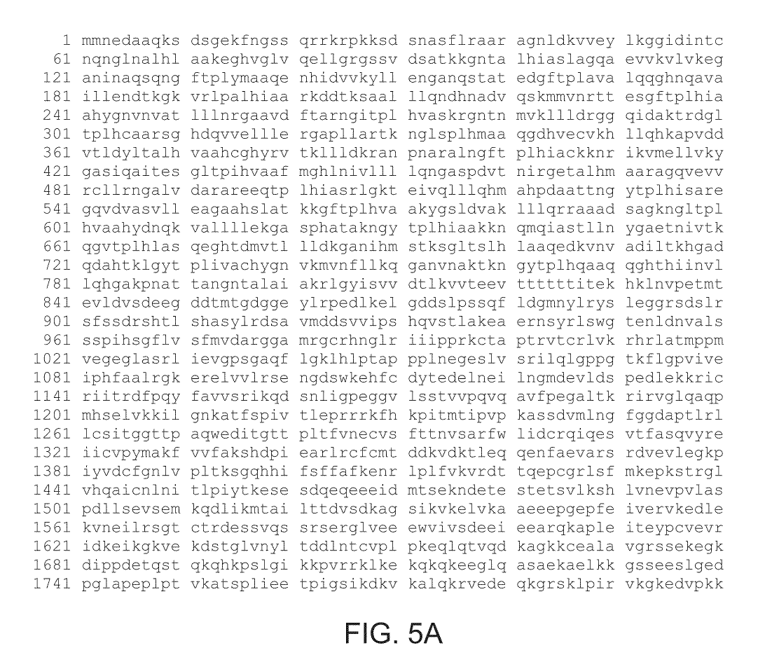

Alternative splicing results in several transcript variants encoding different isoforms. An amino acid sequence of Homo sapiens isoform-a is presented in GenBank Accession No. Q14524; FIG. 4; and Gellens et al. (1992) Proc. Natl. Acad. Sci. USA 89:554. GenBank Accession No. NP_{—}000326 provides an amino acid sequence of Homo sapiens SCN5A isoform-b (2015 amino acids). The mRNA variant encoding isoform-b uses a different acceptor splice site at one of the coding exons, 3 nucleotides downstream of that used by transcript variant 1 encoding isoform-a. This results in an isoform (b) shorter by just a single amino acid, compared to isoform-a. GenBank Accession No. NP_{—}001092874 provides an amino acid sequence of Homo sapiens SCN5A isoform-c (2016 amino acids). The mRNA variant encoding isoform-c uses an alternate, duplicated coding exon compared to the transcript variant encoding isoform-a, resulting in an isoform (c) of the same size, but differing in a few internal amino acids compared to isoform-a. GenBank Accession No. NP_{—}001092875 provides an amino acid sequence of Homo sapiens SCN5A isoform-d (1998 amino acids). The mRNA variant encoding isoform-d uses an alternate, duplicated coding exon, and is missing another in-frame, downstream coding exon compared to transcript variant 1 encoding isoform-a, resulting in a shorter isoform (d) missing an internal segment and differing in a few amino acids, compared to isoform-a.

Mutations of SCN5A that are associated with LQTS are known in the art. For example, Wei et al. ((1999) Circulation 99:3165 reported an E1784K substitution associated with LQTS; Makita et al. ((2002) Circulation 106:1269) reported an L1825P missense mutation associated with LQTS; Wang et al. ((2004) J. Med. Genet. 41:e66) reported an R1193Q mutation associated with acquired LQTS; the mutations N1325S and R1644H have been reported to be associated with LQTS.

Mutations of SCN5A that are associated with LQTS are in some embodiments encoded by a mutant of the nucleotide sequence identified as SEQ ID NO:3 in U.S. Pat. No. 6,787,309, where the mutant results from one or more of a G3340A substitution, a C4501G substitution, a deletion of nucleotides 4850-4852, a G4868T substitution, and a G5360A substitution, compared to SEQ ID NO:3 of U.S. Pat. No. 6,787,309. U.S. Pat. No. 6,787,309 reports that mutations in SCN5A such as D1114N, L1501V, deletion of F1617, R1623L, and S1787N are associated with LQTS. See, e.g., Table 4 of U.S. Pat. No. 6,787,309, which is reproduced below as Table 3, and depicts mutations compared to the amino acid sequence depicted in FIG. 4.

TABLE 3

| Nucleotide change | Amino acid change |
|---|---|
| G3340A | D1114N |
| C3911T | T1304M |
| A3974G | N1325S |
| C4501G | L1501V |
| del4511-4519 | del1505-1507 |
| del4850-4852 | delF1617 |
| G4868A | R1623Q |
| G4868T | R1623L |
| G4931A | R1644H |
| C4934T | T1645M |
| G5350A | E1784K |
| G5360A | S1787N |
| A5369G | D1790G |
| insTGA 5385-5386 | insD1795-1796 |

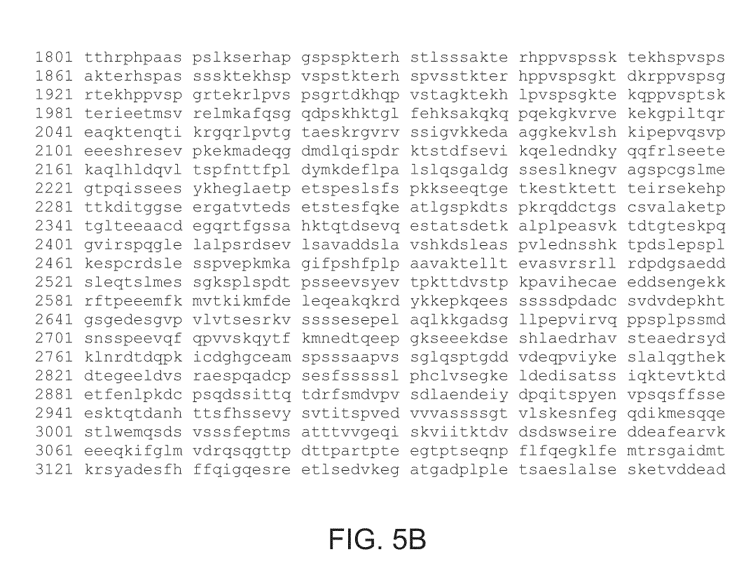

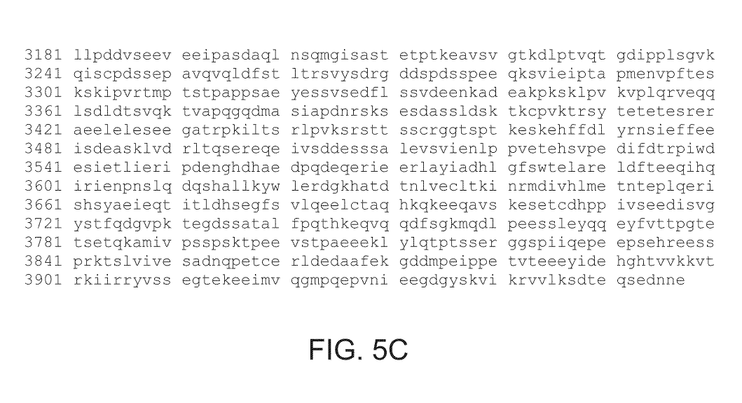

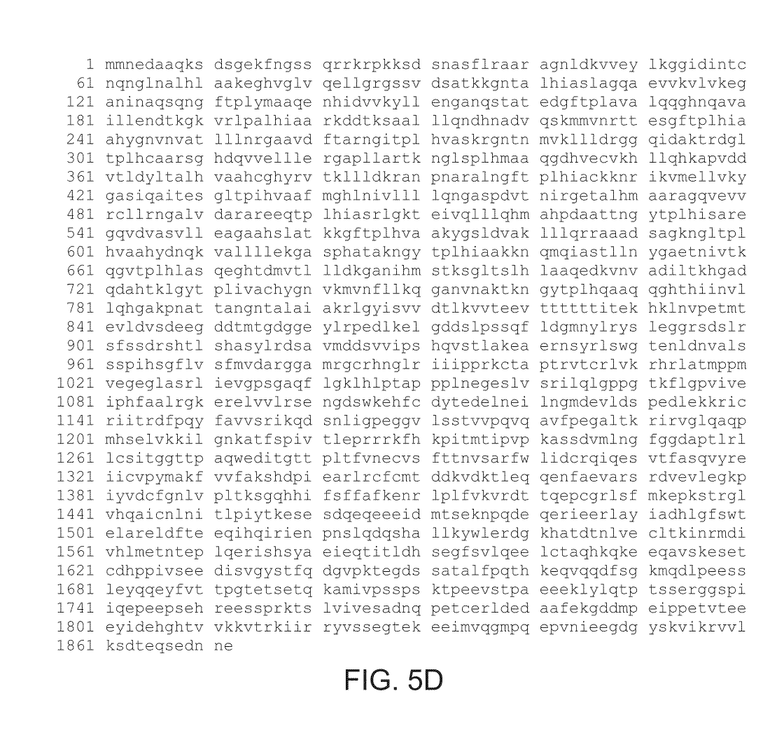

ANK2 is a gene that encodes an ankyrin-2 (ANK2) polypeptide, a cytoskeletal protein that interacts with ion channels. Mutations in the ankyrin-B-encoding gene (ANK2) can cause type 4 long-QT syndrome. An amino acid sequence of Homo sapiens ANK2 wild-type isoform 1 is presented in GenBank Accession No. NP_{—}001139) and in FIGS. 5A-C; an amino acid sequence of Homo sapiens ANK2 wild-type isoform 2 is presented in GenBank Accession No. NP_{—}066187, and FIG. 5D. ANK2 variants associated with LQTS (in particular, LQT4) are known in the art.

See, e.g., Mank-Seymour et al. (2006) Am. Heart J. 152:1116; Mohler et al. (2007) Circulation 115:432. ANK2 variants T14041, V1516D, T1552N, V1777M, and E1813K at equivalent levels were found to display abnormal contraction rates and aberrant spatial/temporal patterns of Ca²⁺ release; and loss-of-function variants E1425G, V1516D, and R1788W were associated with severe arrhythmias. Mohler et al. (2007) Circulation 115:432. Other ankyrin-B variants include L16221, T1626N, and R1788W.

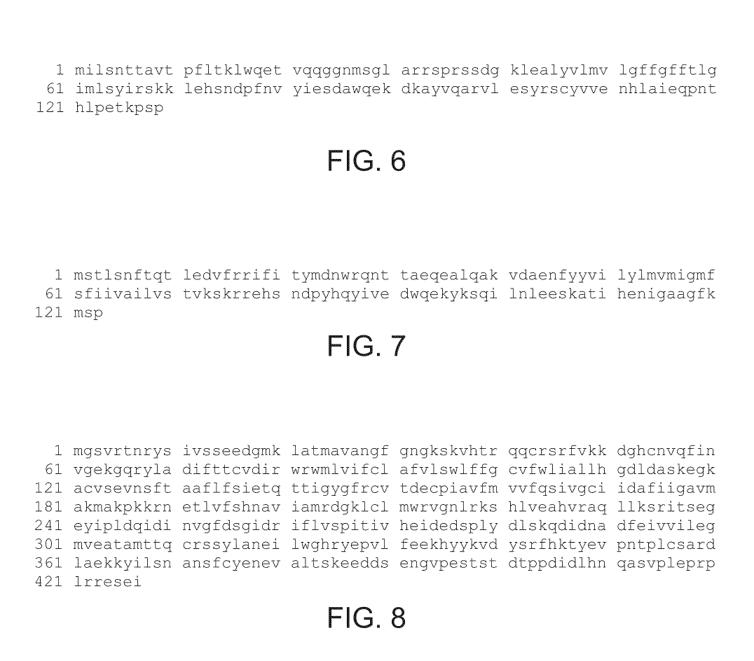

KCNE1 encodes the MinK protein, a cardiac K-channel accessory subunit. KCNE1 (LQT5) mutations account for approximately of 2-3% of genotyped LQTS patients and may cause both Romano-Ward (LQT5) and, if homozygous, Jervell and Lange-Nielsen (JLN2). Amino acid sequences of Homo sapiens MinK polypeptide are known in the art; see, e.g., GenBank Accession Nos. CAG46556, NP_{—}000210 (and FIG. 6), AAH46224, and P15382.

MinK variants associated with LQTS (e.g., LQT5) include, e.g., A8V and R98W (Ohno et al. (2007) Heart Rhythm 4:332); and the variants depicted in Table 5 of U.S. Pat. No. 6,787,309 (e.g., T71; R32H; V47F; L51H; TL58-59PP; S74L; K76N; W87R; R98W; and P127T). See, Table 4, below.

TABLE 4

| Nucleotide change | Amino acid change |
|---|---|
| C20T | T7I |
| G95A | R32H |
| G139T | V47F |
| TG151-152AT | L51H |
| A172C/TG 176-177CT | TL58-59PP |
| C221T | S74L |
| G226A | D76N |
| T259C | W87R |
| C292T | R98W |
| C379A | P127T |

MiRP1 (encoded by KCNE2) a member of the potassium channel, voltage-gated, isk-related subfamily. MiRP1 is a small integral membrane subunit that assembles with the KCNH2 gene product, a pore-forming protein, to alter its function. The MiRP1 gene is expressed in heart and muscle and gene mutations are associated with cardiac arrhythmia. Amino acid sequences of Homo sapiens MiRP1 polypeptide are known in the art; see, e.g., GenBank Accession Nos. AAD28086, NP_{—}751951 (and FIG. 7), AAH93892, and AA112088.

Mutations in MiRP1 that are associated with LQTS (e.g., LQT6)) include T8A, Q9E, M54T, 157T, and A116V. See, e.g., Sesti et al. (2000) Proc. Natl. Acad. Sci. USA 97:10613; and Lu et al. (2003) J. Physiol. 551.1:253.

Kir2.1 is an inward rectifier K⁺ channel encoded by the KCNJ2 gene. Mutations in KCNJ2 have been identified in patients with Andersen-Tawil syndrome (ATS; also known as LTQ7). Amino acid sequences of Homo sapiens Kir2.1 polypeptides are known in the art; see, e.g., GenBank Accession Nos. NP_{—}000882 (and FIG. 8), and P63252.

Mutations of KCNJ2 that are associated with LQTS are known in the art. See, e.g., Tristani-Firouzi et al. (2002) J. Clin. Invest. 110:381; Fodstad et al. (2004) J. Mol. Cell. Cardiol. 37:593; Eckhardt et al. (2007) Heart Rhythm 4:323; Bendahhou et al. (2007) Hum. Mol. Genet. 16:900. Examples of mutations in the encoded Kir2.1 polypeptide include, e.g., C54F, T75A, P186L, V302M, T305A, T305P, and N216H.

CACNA1c encodes Cav1.2, an alpha-1 subunit of a voltage-dependent calcium channel. Amino acid sequence of Homo sapiens CACNA1c polypeptides are known in the art. See, e.g., GenBank Accession No. NP_{—}000710. Amino acid sequences of Homo sapiens CACNA1c soforms CRA_a through CRA_p are found under GenBank Accession Nos. EAW88895 (isoform CRA_a); EAW88896 (isoform CRA_b); EAW88897 (isoform CRA_c); EAW88898 (isoform CRA_d); EAW88899 (isoform CRA_e); EAW88900 (isoform CRA_f); EAW888901 (isoform CRA_g); EAW888902 (isoform CRA_h); EAW888903 (isoform CRA_i); EAW888904 (isoform CRA_j); EAW888905 (isoform CRA_k); EAW888906 (isoform CRA_{—}1); EAW888907 (isoform CRA_m); EAW888908 (isoform CRA_n); EAW888909 (isoform CRA_o); and EAW888910 (isoform CRA_p).

Mutations in CACNA1c that are associated with LTQS (e.g., LQT8 or Timothy syndrome) are known in the art. Splawski et al. (2005) Proc. Natl. Acad. Sci. USA 102:8089. Mutations include, e.g., G406R. Splawski et al. (2004) Cell 119:19.

CAV3 encodes caveolin 3. Amino acid sequences of Homo sapiens caveolin-3 are known in the art; see, e.g., GenBank Accession Nos. NP_{—}001225 and NP_{—}203123. Mutations in caveolin-3 are associated with LQT9. Mutations in caveolin-3 that have been associated with LQTS include V14L, T78M, and L79R.

SCN4B encodes Navβ4, a sodium channel β subunit. Amino acid sequences of Homo sapiens SCN4B are known in the art; see, e.g., GenBank Accession Nos. NP_{—}777594, AAN74584, and Q81WT1. An L179F missense mutation in the protein encoded by SCN4B is associated with LQTS. Medeiros-Domingo et al. (2007) Circulation 116:134. Mutations in SCN4B are associated with LQT10.

Drugs that are known to induce LQTS in some individuals include, but not limited to: 1) antibiotics such as erythromycin and other macrolides, fluoroquinolones, trimethoprim, sulfamethoxazole, halofantrine, and pentamidine; 2) antihistamines such as seldane (terfenadine), hismanal (astemizole), and Benadryl (diphenhydramine); 3) heart medications, such as quinidine, pronestyl, procainamide, disopyramide, dofetilide, sotalol, ibutilide, probucol, and bepridil; 4) anti-fungal agents such as ketoconazole, fluconazole, itraconazole; 5) psychotropic drugs such as amitryptiline, fluoxetine, venlafaxine, roxetine, phenothiazine derivatives, haloperidol, risperidone, quetiapine, ziprasedone, sertraline, thioridazine, levomethadyl, mesoridazine, and pimozide; 6) blood pressure medication such as indapamide, nicardipine, moexipril and isradipine; 7) cancer medication such as arsenic trioxide, tamoxifen; 8) anti viral drugs such as foscarnet; 9) drugs for treating neurological diseases, e.g., felbamate; fosphenyloin; and selective serotonin agonists such as naratriptan, sumatriptan and zolmitriptan; 10) anti-emesis drugs such as dolasetron, and droperidol; 11) muscle relaxants such as tizanidine; 12) drugs for treating pulmonary diseases, e.g., salmeterol; 13) drugs for treating endocrinological diseases, e.g., octreotide; 14) immunosuppressive medication such as tacrolimus; and 15) medication for gastric stimulation such as cisapride.

### Cell Panels

The present disclosure further provides panels of cardiomyocytes suitable for use in screening compounds for an effect on the QT interval. A subject cell panel can include one or more cells of the following cell types: 1) a cardiomyocyte generated from a cell of an individual having no known mutations in a gene associated with LQTS or any polypeptide that is associated with control of the QT interval, where the individual has never had an LQTS episode; 2) a cardiomyocyte generated from a cell from an individual having one or more mutations in an LQTS-associated gene, where the one or more mutations are known to be associated with increased risk of LQTS; and 3) a cardiomyocyte generated from a cell from an individual who has had at least one LQTS episode, but who has no known LQTS-associated mutations. In particular embodiments, the cardiomyocytes are human cardiomyocytes. In some embodiments, a cardiomyocyte included in a subject panel is generated from an iPS cell or an iMS cell generated from a somatic cell obtained from an individual, e.g., a human (e.g., a post-natal human).

As discussed above, in some embodiments, a cardiomyocyte is generated from an iPS cell or an iMS cell. In addition to iPS cells and iMS cells, any pluripotent, multipotent, or other non-lineage-committed cell can be used to generate a cardiomyocyte. In some embodiments, the cell used to generate a cardiomyocyte is any multipotent or pluripotent cell other than an embryonic stem (ES) cell.

“LQTS-associated gene” and “gene encoding a polypeptide that controls the QT interval” are used interchangeably herein to refer to genes that encode polypeptides that control (directly or indirectly) the QT interval and that, when mutated, encode a mutated polypeptide that has altered function, e.g., has a lengthening effect on the QT interval and can cause LQTS. Examples of LQTS-associated genes include KCNQ1, KCNH2, SCN5A, Ank2, KCNE1, KCNE2, KCNJ2, CACNA1C, CAV3, and SCN4B. Examples of polypeptides that, when mutated, can lengthen the QT interval and can result in LQTS in an individual, include, e.g., KVLQT1, HERG, Nav1.5, Ankyrin B, mink, miRP1, Kir2.1, Cav1.2, caveolin-3, and Navβ4.

Cardiomyocytes in a subject panel can be generated from iPS cells, other pluripotent cells, or iMS cells, as described above. Thus, a “cardiomyocyte generated from an iPS cell or an iMS cell from an individual” is a cardiomyocyte that has been induced to differentiate in vitro from an iPS cell or an iMS cell that has been generated from a cell (e.g., a somatic cell) obtained from an individual (e.g., a post-natal human). In other words, a cardiomyocyte that is suitable for use in a subject panel includes a cardiomyocyte that is generated by: 1) inducing a somatic cell, obtained from an individual, to become a pluripotent stem cell, thereby generating an iPS cell or an iMS cell; and 2) inducing the iPS cell or iMS cell to undergo cardiomyogenesis in vitro, thereby generating a cardiomyocyte from a somatic cell obtained from an individual.

A subject cell panel can include from 5 different cardiomyocytes to about 1000 different cardiomyocytes, e.g., from about 5 different cardiomyocytes to about 10 different cardiomyocytes, from about 10 different cardiomyocytes to about 20 different cardiomyocytes, from about 20 different cardiomyocytes to about 25 different cardiomyocytes, from about 25 different cardiomyocytes to about 50 different cardiomyocytes, from about 50 different cardiomyocytes to about 75 different cardiomyocytes, from about 75 different cardiomyocytes to about 100 different cardiomyocytes, from about 100 different cardiomyocytes to about 250 different cardiomyocytes, from about 250 different cardiomyocytes to about 500 different cardiomyocytes, or from about 500 different cardiomyocytes to about 1000 different cardiomyocytes. In some embodiments, a subject cell panel can include more than 1000 different cardiomyocytes. A subject cell panel can also comprise fewer than 5 different cardiomyocytes, e.g., a subject cell panel can include 2, 3, or 4 cardiomyocytes.

A subject cell panel can include cardiomyocytes generated from cells obtained from about 5 individuals to about 1000 individuals, or more than 1000 individuals. A subject cell panel can include cardiomyocytes obtained from about 5 individuals to about 25 individuals, from about 25 different individuals to about 50 different individuals, from about 50 different individuals to about 75 different individuals, from about 75 different individuals to about 100 different individuals, from about 100 different individuals to about 250 different individuals, from about 250 different individuals to about 500 different individuals, or from about 500 different individuals to about 1000 different individuals. In some embodiments, a subject cell panel can include cells derived from more than 1000 different individuals. A subject cell panel can also comprise cardiomyocytes obtained from or derived from fewer than 5 individuals, e.g., 2 individuals, 3 individuals, or 4 individuals.

A subject cell panel can comprise cells in any of a variety of formats. For example, cells can be present in wells of a multi-well plate, where each different cardiomyocyte is present in separate wells. A cardiomyocyte can be present in a well of a multi-well plate, where the cardiomyocyte is present in the well at from about 10 cells per well to about 10⁶ cells per well, or more than 10⁶ cells per well. Multi-well plates can be 6-well, 12-well, 24-well, or 96-well plates, or the plates can include more than 96 wells (e.g., multiples of 96).

### Normal Cardiomyocytes

As noted above, a subject cardiomyocyte panel can include a cardiomyocyte generated from a cell of an individual having no known mutations in a gene associated with LQTS or any polypeptide that is associated with control of the QT interval, where the individual has never had an LQTS episode. Such cardiomyocytes are referred to herein as “normal cardiomyocytes.” For example, in some embodiments, “normal cardiomyocytes” do not have any mutations in polypeptides that are associated with control of the QT interval, and exhibit normal electrical behavior in vitro, e.g., exhibit a normal QT interval in vitro.

The source of normal cardiomyocytes can include individuals of a particular population or sub-population, e.g.: 1) individuals of a particular genetic background, e.g., individuals of a particular major histocompatibility complex (MHC) (or human leukocyte antigen; HLA) haplotype, etc.; 2) individuals of a particular race, e.g., Caucasian individuals; African individuals or individuals of African descent; Asian individuals or individuals of Asian descent; native American individuals; African-Americans; Hispanic/Latino individuals; etc.; 3) female individuals; 4) male individuals; 5) individuals having a particular disease state, e.g., individuals with chronic kidney disease such as end-stage renal failure; individuals with known cardiovascular disease; individuals with liver disease; etc.; 6) individuals known to have been exposed chronically to an environmental toxin; 7) individuals who are habitual smokers of tobacco products (e.g., cigarettes); 8) individuals who are considered to be heavy consumers of alcohol; 9) individuals of a particular national or geographic origin; and the like.

In some embodiments, a subject cardiomyocyte panel includes from about 5 different normal cardiomyocytes to about 10 different normal cardiomyocytes, from about 10 different normal cardiomyocytes to about 20 different normal cardiomyocytes, from about 20 different normal cardiomyocytes to about 25 different normal cardiomyocytes, from about 25 different normal cardiomyocytes to about 50 different normal cardiomyocytes, from about 50 different normal cardiomyocytes to about 75 different normal cardiomyocytes, from about 75 different normal cardiomyocytes to about 100 different normal cardiomyocytes, from about 100 different normal cardiomyocytes to about 250 different normal cardiomyocytes, from about 250 different normal cardiomyocytes to about 500 different normal cardiomyocytes, or from about 500 different normal cardiomyocytes to about 1000 different normal cardiomyocytes. In some embodiments, a subject cell panel can include more than 1000 different normal cardiomyocytes.

In some embodiments, a subject cardiomyocyte panel includes a first cardiomyocyte derived from an individual of a first HLA haplotype; and at least a second cardiomyocyte derived from an individual of a second HLA haplotype. For example, in some embodiments, a subject cardiomyocyte panel includes a first cardiomyocyte derived from an individual of a first HLA haplotype; a second cardiomyocyte derived from an individual of a second HLA haplotype; a third cardiomyocyte derived from an individual of a third HLA haplotype; a fourth cardiomyocyte derived from an individual of a fourth HLA haplotype; a fifth cardiomyocyte derived from an individual of a fifth HLA haplotype; and optionally additional cardiomyocytes derived from individuals of additional HLA haplotypes.

In some embodiments, a subject cardiomyocyte panel includes a first cardiomyocyte derived from a Caucasian individual; a second cardiomyocyte derived from an African-American individual; a third cardiomyocyte derived from an Asian individual; a fourth cardiomyocyte derived from an Hispanic/Latino individual; etc.

In some embodiments, a subject cardiomyocyte panel includes a first cardiomyocyte derived from a Caucasian individual of a first HLA haplotype; a second cardiomyocyte derived from a Caucasian individual of a second HLA haplotype; a third cardiomyocyte derived from a Caucasian individual of a third HLA haplotype; a fourth cardiomyocyte derived from a Caucasian individual of a fourth HLA haplotype; and optionally additional cardiomyocytes derived from Caucasian individuals of additional HLA haplotypes; a fifth cardiomyocyte derived from an Asian individual of a first HLA haplotype; a sixth cardiomyocyte derived from an Asian individual of a second HLA haplotype; a seventh cardiomyocyte derived from an Asian individual of a third HLA haplotype; and optionally additional cardiomyocytes derived from Asian individuals of additional HLA haplotypes; an eighth cardiomyocyte derived from an Hispanic/Latino individual of a first HLA haplotype; a ninth cardiomyocyte derived from an Hispanic/Latino individual of a second HLA haplotype; a tenth cardiomyocyte derived from an Hispanic/Latino individual of a third HLA haplotype; and optionally additional cardiomyocytes derived from Hispanic/Latino individuals of additional HLA haplotypes; an eleventh cardiomyocyte derived from an African-American individual of a first HLA haplotype; a twelfth cardiomyocyte derived from an African-American individual of a second HLA haplotype; a thirteenth cardiomyocyte derived from an African-American individual of a third HLA haplotype; and optionally additional cardiomyocytes derived from African-American individuals of additional HLA haplotypes. Those skilled in the art will appreciate that additional cardiomyocytes derived from individuals of various populations and sub-populations can be included in such a cardiomyocyte panel.

Cardiomyocytes from Individuals Who have No Known LQTS-Associated Mutation and Who have Had at Least One LQTS Episode

As noted above, a subject cardiomyocyte panel can include a cardiomyocyte generated from a cell of an individual having no known mutations in a gene associated with LQTS or any polypeptide that is associated with control of the QT interval, where the individual has had at least one LQTS episode.

In some embodiments, a subject cardiomyocyte panel includes a cardiomyocyte generated from an iPS or an iMS cell generated from a somatic cell from an individual who has experienced LQTS (either non-drug-induced LQTS or drug-induced LQTS) and whose genotype with respect to an LQTS-associated gene, such as HERG, is unknown.

In some embodiments, a subject cardiomyocyte panel includes a cardiomyocyte derived from an individual (e.g., generated from an iPS cell or an iMS cell generated from a cell from the individual) who has had at least one LQTS episode, where the at least one LQTS episode was not drug induced, and where the individual has no known LQTS-associated mutation.

In some embodiments, a subject cardiomyocyte panel includes a cardiomyocyte derived from an individual (e.g., generated from an iPS cell or an iMS cell generated from a cell from the individual) who has had at least one LQTS episode, where the at least one LQTS episode was drug induced, and where the individual has no known LQTS-associated mutation.

In some embodiments, a subject cardiomyocyte panel includes a cardiomyocyte derived from an individual (e.g., generated from an iPS cell or an iMS cell generated from a cell from the individual) who has had at least one drug-induced LQTS episode, where the individual has no known LQTS-associated mutation, and where the drug that induced the LQTS was a drug known to have the potential to induce LQTS.

Drugs that are known to have the potential to induce LQTS include, but are not limited to, 1) antibiotics such as erythromycin, clarithromycin, and other macrolides, fluoroquinolones, sparfloxacin, sulfamethoxazole, trimethoprim, sulfamethoxazole, halofantrine, and pentamidine; 2) antihistamines such as seldane (terfenadine), hismanal (astemizole), azelastine, clemastine, and Benadryl (diphenhydramine); 3) heart medications, such as quinidine, pronestyl, procainamide, disopyramide, dofetilide, sotalol, ibutilide, probucol, bepridil, amiodarone, sotalol, flecamide, moricizine, and tocamide; 4) anti-fungal agents such as ketoconazole, fluconazole, itraconazole; 5) psychotropic drugs such as amitryptiline, amitryptiline-HCl, amoxapine, desipramine, doxepin, fluvoxamine, imipramine, maprotiline, nortryptiline, fluoxetine, venlafaxine, roxetine, perphenazine, chlorpromazine, clomipramine, fluphenazine, thiothixene, trifluoperazine, phenothiazine derivatives, haloperidol, risperidone, quetiapine, ziprasedone, sertraline, thioridazine, levomethadyl, mesoridazine, and pimozide; 6) blood pressure medication such as indapamide, nicardipine, moexipril, fludrocortisones, and isradipine; 7) cancer medication such as arsenic trioxide, tamoxifen; 8) anti viral drugs such as foscamet; 9) drugs for treating neurological diseases, e.g., felbamate; fosphenyloin; and selective serotonin agonists such as naratriptan, sumatriptan and zolmitriptan; 10) anti-emetic drugs such as dolasetron, prochlorperazine, and droperidol; 11) muscle relaxants such as tizanidine; 12) drugs for treating pulmonary diseases, e.g., salmeterol; 13) drugs for treating endocrinological diseases, e.g., octreotide; 14) immunosuppressive medication such as tacrolimus; and 15) medication for gastric stimulation such as cisapride.

In some embodiments, a subject cardiomyocyte panel includes a cardiomyocyte derived from an individual (e.g., generated from an iPS cell or an iMS cell generated from a cell from the individual) who has had at least one drug-induced LQTS episode, where the individual has no known LQTS-associated mutation, where the drug that induced the LQTS was a drug known to have the potential to induce LQTS, and where the drug is a psychotropic drug, e.g., an anti-psychotic drug.

In some embodiments, a subject cardiomyocyte panel includes a cardiomyocyte derived from an individual (e.g., generated from an iPS cell or an iMS cell generated from a cell from the individual) who has had at least one drug-induced LQTS episode, where the individual has no known LQTS-associated mutation, where the drug that induced the LQTS was a drug known to have the potential to induce LQTS, and where the drug is an antibiotic.

In some embodiments, a subject cardiomyocyte panel includes a cardiomyocyte derived from an individual (e.g., generated from an iPS cell or an iMS cell generated from a cell from the individual) who has had at least one drug-induced LQTS episode, where the individual has no known LQTS-associated mutation, where the drug that induced the LQTS was a drug known to have the potential to induce LQTS, and where the drug is a cancer chemotherapeutic agent.

In some embodiments, a subject cardiomyocyte panel includes a cardiomyocyte derived from an individual (e.g., generated from an iPS cell or an iMS cell generated from a cell from the individual) who has had at least one drug-induced LQTS episode, where the individual has no known LQTS-associated mutation, where the drug that induced the LQTS was a drug known to have the potential to induce LQTS, and where the drug is an antihistamine.

Cardiomyocytes Comprising at Least One Mutation Known to be Associated with Increased Risk of LQTS

Where a cardiomyocyte comprises at least one LQTS-associated mutation in an LQTS-associated gene, the mutation is associated with a LQTS wherein the Q-T interval corrected for heart rate (QTc) is longer than 440 millisecond (ms), longer than 450 millisecond, or longer than 460 millisecond. QTc can be calculated using the following formula: QTc=QT/(square root of the R−R interval). This is also known as Bazett's formula. Bazett (1920) Heart 7:353-370. The R−R interval is the interval from the onset of one QRS complex to the onset of the next QRS complex. The normal QT interval is from about 330 ms to about 440 ms (or about 450 ms for women). In some embodiments, the mutation alters a function of the encoded potassium channel, sodium channel, or calcium channel, where the alteration can be detected using a dye that provides a detectable signal in response to changes in intracellular ion (e.g., sodium, potassium, or calcium) concentration.

In some embodiments, a subject cardiomyocyte panel includes at least a first cardiomyocyte and a second cardiomyocyte, where the first cardiomyocyte is generated by inducing an induced pluripotent stem cell obtained from a somatic cell from a first individual to differentiate into a cardiomyocyte in vitro, where the first cardiomyocyte comprises a first mutation in a first polypeptide that controls the QT interval, where the first mutation results in LQTS (e.g., the first mutation is known to be associated with higher risk of LQTS); where the second cardiomyocyte is generated by inducing an induced pluripotent stem cell obtained from a somatic cell from a second individual to differentiate into a cardiomyocyte in vitro, where the second cardiomyocyte comprises a the first mutation in the first polypeptide that controls the QT interval, e.g., the second cardiomyocyte comprising the same LQTS-associated mutation as the first cardiomyocyte. In some of these embodiments, the first cardiomyocyte is from a first individual, and the second cardiomyocyte is from a second individual, where the first and the second individuals are of different populations or sub-populations. For example, in some embodiments, the first individual is of a first race and the second individual is of a second race. As another example, the first individual is a known habitual smoker of a tobacco product (e.g., cigarettes), and the second individual is a known non-smoker of tobacco products. In some embodiments, the panel further includes a cardiomyocyte from in individual who has no known LQTS-associated mutation.

In some embodiments, a subject cardiomyocyte panel includes at least a first cardiomyocyte and a second cardiomyocyte, where the first cardiomyocyte is generated by inducing an induced pluripotent stem cell obtained from a somatic cell from a first individual to differentiate into a cardiomyocyte in vitro, where the first cardiomyocyte comprises a first mutation in a first polypeptide that controls the QT interval, where the first mutation is associated with long QT syndrome (LQTS); and where the second cardiomyocyte is generated by inducing an induced pluripotent stem cell obtained from a somatic cell from a second individual to differentiate into a cardiomyocyte in vitro. In some embodiments, the first polypeptide is selected from HERG, KvLQT1, Nav1.5, ankyrin-B, MinK, MiRP1, Kir2.1, Cav1.2, caveolin-3, and Navβ4. In some embodiments, the second cardiomyocyte does not comprise an LQTS-associated mutation in polypeptide that controls the QT interval, e.g., the second cardiomyocyte is a “normal” control. In some embodiments, the second cardiomyocyte comprises the same LQTS-associated mutation as the first cardiomyocyte. For example, where the second cardiomyocyte comprises the same LQTS-associated mutation as the first cardiomyocyte, the first and the second cardiomyocytes can be from individuals of different populations or sub-populations. In some embodiments, the second cardiomyocyte comprises a second mutation in the first polypeptide, where the second mutation is an LQTS-associated mutation, e.g., the second cardiomyocyte comprises a different mutation in the same LQTS-associated polypeptide in which the mutation in the first cardiomyocyte is found. For example, the second cardiomyocyte could include an R243c substitution in KVLQT1, and the first cardiomyocyte could include a T311I substitution in KVLQT1. As another example, the second cardiomyocyte could include an F29L substitution in HERG, and the first cardiomyocyte could include an H70R substitution in HERG. In other embodiments, the second cardiomyocyte comprises a first mutation in a second polypeptide that controls the QT interval, where the first mutation in the second polypeptide is an LQTS-associated mutation. For example, the second cardiomyocyte could include an R243c substitution in KVLQT1, and the first cardiomyocyte could include an H70R substitution in HERG. As another example, the second cardiomyocyte could include an F29L substitution in HERG, and the first cardiomyocyte could include a T311I substitution in KVLQT1. A subject panel will in some embodiments include a third cardiomyocyte, where the third cardiomyocyte is generated by inducing an induced pluripotent stem cell obtained from a somatic cell from a third individual to differentiate into a cardiomyocyte in vitro. A subject panel can include a fourth, fifth, sixth, seventh, eighth, etc. cardiomyocyte.

In some embodiments, a subject cardiomyocyte panel includes at least a first cardiomyocyte and a second cardiomyocyte, where the first cardiomyocyte is generated by inducing an induced pluripotent stem cell obtained from a somatic cell from a first individual to differentiate into a cardiomyocyte in vitro, where the first cardiomyocyte comprises a first mutation in a first polypeptide that controls the QT interval, where the first mutation results in LQTS (e.g., the first mutation is known to be associated with higher risk of LQTS); where the second cardiomyocyte is generated by inducing an induced pluripotent stem cell obtained from a somatic cell from a second individual to differentiate into a cardiomyocyte in vitro, where the second cardiomyocyte comprises a second mutation in the first polypeptide that controls the QT interval, and where the second mutation results in LQTS (e.g., the second mutation is known to be associated with higher risk of LQTS).

In some embodiments, a subject cardiomyocyte panel includes: 1) at least a first cardiomyocyte comprising a first LQTS-associated mutation in a first LQTS-associated gene (e.g., KCNQ1, KCNH2, SCN5A, Ank2, KCNE1, KCNE2, KCNJ2, CACNA1C, CAV3, SCN4B, etc.); and 2) at least a second cardiomyocyte comprising a second LQTS-associated mutation in the first LQTS-associated gene, where the first and the second mutations are not the same. In some of these embodiments, the panel further includes a cardiomyocyte that does not comprise any LQTS-associated mutations in any LQTS-associate genes, and does not exhibit LQTS. In some of these embodiments, the panel will further include one or more additional cardiomyocytes comprising either additional mutations in the first LQTS-associated gene, or mutations in an LQTS-associated gene other than the first LQTS-associated gene.

In other embodiments, a subject cardiomyocyte panel includes: 1) at least a first cardiomyocyte comprising an LQTS-associated mutation in a first LQTS-associated gene; and 2) at least a second cardiomyocyte comprising an LQTS-associated mutation in a second LQTS-associated gene, where the first and the second LQTS-associated genes are not the same. The first and the second LQTS-associated genes can be selected from KCNH2, KCNQ1, SCN5A, ANK2, KCNE1, KCNE2, KCNJ2, CACNA1c, CAV3, and SCN4B; and the cardiomyocyte can produce a mutated LQTS-associated polypeptide selected from HERG, KvLQT1, Nav1.5, ankyrin-B, MinK, Kir2.1, CAv1.2, caveolin-3, and Navβ4. In some of these embodiments, the panel further includes a cardiomyocyte that does not comprise any LQTS-associated mutations in any LQTS-associate genes, and does not exhibit LQTS. In some of these embodiments, the panel will further include one or more additional cardiomyocytes comprising either mutations in one or more additional LQTS-associated genes other than the first and the second LQTS-associated genes, or additional mutations in the first and/or the second LQTS-associated genes.

In some embodiments, a subject cardiomyocyte panel includes: 1) at least a first cardiomyocyte comprising a first LQTS-associated mutation in a first LQTS-associated gene; 2) at least a second cardiomyocyte comprising a second LQTS-associated mutation in the first LQTS-associated gene; 3) at least a third cardiomyocyte comprising a first LQTS-associated mutation in a second LQTS-associated gene; and 4) at least a fourth cardiomyocyte comprising a second LQTS-associated mutation in the second LQTS-associated gene. The first and the second LQTS-associated genes can be selected from KCNH2, KCNQ1, SCN5A, ANK2, KCNE1, KCNE2, KCNJ2, CACNA1c, CAV3, and SCN4B; and the cardiomyocyte can produce a mutated LQTS-associated polypeptide selected from HERG, KvLQT1, Nav1.5, ankyrin-B, MinK, Kir2.1, CAv1.2, caveolin-3, and Navβ4. In some of these embodiments, the panel further includes a cardiomyocyte that does not comprise any LQTS-associated mutations in any LQTS-associate genes, and does not exhibit LQTS. In some of these embodiments, the panel will further include one or more additional cardiomyocytes comprising either additional mutations in a given LQTS-associated gene, or comprising mutations in a further LQTS-associated gene.

In some embodiments, a subject cardiomyocyte panel includes: 1) at least a first cardiomyocyte comprising an LQTS-associated mutation in KCNH2 encoding HERG; 2) at least a second cardiomyocyte comprising a wild-type KCNH2 encoding wild-type HERG, e.g., a KCNH2 gene that does not include an LQTS-associated mutation. For example, in some embodiments, a subject cardiomyocyte panel includes a cardiomyocyte comprising an LQTS-associated mutation in HERG depicted in Table 3 of U.S. Pat. No. 6,787,309 (or Table 2, above).

For example, in some embodiments, a subject cardiomyocyte panel includes a cardiomyocyte comprising an LQTS-associated mutation in HERG, e.g., the panel include one or more of: 1) a cardiomyocyte generated from an iPS or iMS cell from an individual comprising a C87A mutation that results in an F29L substitution in the encoded HERG polypeptide; 2) a cardiomyocyte generated from an iPS or iMS cell from an individual comprising an A98C mutation that results in a C44X mutation in the encoded HERG polypeptide; 3) a cardiomyocyte generated from an iPS or iMS cell from an individual comprising a G140T mutation that results in a G47V mutation in the encoded HERG polypeptide; 4) a cardiomyocyte generated from an iPS or iMS cell from an individual comprising aG157C mutation resulting a G53R substitution in the encoded HERG polypeptide; 5) a cardiomyocyte generated from an iPS or iMS cell from an individual comprising a G167A mutation that results in an R56Q substitution in the encoded HERG polypeptide; 6) a cardiomyocyte generated from an iPS or iMS cell from an individual comprising a T196G mutation that results in a C66G substitution in the encoded HERG polypeptide; 7) a cardiomyocyte generated from an iPS or iMS cell from an individual comprising an A209G mutation that results in an H70R substitution in the encoded HERG polypeptide; 8) a cardiomyocyte generated from an iPS or iMS cell from an individual comprising a C215A mutation that results in a P72Q substitution in the encoded HERG polypeptide; 9) a cardiomyocyte generated from an iPS or iMS cell from an individual comprising a G232C mutation that results in an A78P substitution in the encoded HERG polypeptide; 10) a cardiomyocyte generated from an iPS or iMS cell from an individual comprising a T257G mutation that results in an L86R substitution in the encoded HERG polypeptide; 11) a cardiomyocyte generated from an iPS or iMS cell from an individual comprising a C934T mutation that results in an R312c substitution in the encoded HERG polypeptide; 12) a cardiomyocyte generated from an iPS or iMS cell from an individual comprising a C1039T mutation that results in a P347S substitution in the encoded HERG polypeptide; 13) a cardiomyocyte generated from an iPS or iMS cell from an individual comprising a C1283A mutation that results in an S428X substitution in the encoded HERG polypeptide; 14) a cardiomyocyte generated from an iPS or iMS cell from an individual comprising a C1307T mutation that results in a T436M substitution in the encoded HERG polypeptide; 15) a cardiomyocyte generated from an iPS or iMS cell from an individual comprising an A1408G mutation that results in a N470D substitution in the encoded HERG polypeptide; 16) a cardiomyocyte generated from an iPS or iMS cell from an individual comprising a C1421T mutation that results in a T474I substitution in the encoded HERG polypeptide; 17) a cardiomyocyte generated from an iPS or iMS cell from an individual comprising a G1592A mutation that results in a R531Q substitution in the encoded HERG polypeptide; 18) a cardiomyocyte generated from an iPS or iMS cell from an individual comprising a C1600T mutation that results in a R534c substitution in the encoded HERG polypeptide; 19) a cardiomyocyte generated from an iPS or iMS cell from an individual comprising a G1672C mutation that results in an A558P substitution in the encoded HERG polypeptide; 20) a cardiomyocyte generated from an iPS or iMS cell from an individual comprising a G1681A mutation that results in a A561T substitution in the encoded HERG polypeptide; 21) a cardiomyocyte generated from an iPS or iMS cell from an individual comprising a C1682T mutation that results in a A561V substitution in the encoded HERG polypeptide; 22) a cardiomyocyte generated from an iPS or iMS cell from an individual comprising a G1714C mutation that results in a G572R substitution in the encoded HERG polypeptide; 23) a cardiomyocyte generated from an iPS or iMS cell from an individual comprising a C1744T mutation that results in an R582C substitution in the encoded HERG polypeptide; 24) a cardiomyocyte generated from an iPS or iMS cell from an individual comprising a G1750A mutation that results in a G584S substitution in the encoded HERG polypeptide; 25) a cardiomyocyte generated from an iPS or iMS cell from an individual comprising a G1755T mutation that results in a W585C substitution in the encoded HERG polypeptide; 26) a cardiomyocyte generated from an iPS or iMS cell from an individual comprising an A1762G mutation that results in an N588D substitution in the encoded HERG polypeptide; 27) a cardiomyocyte generated from an iPS or iMS cell from an individual comprising a T1778C mutation that results in an 1593T substitution in the encoded HERG polypeptide; 28) a cardiomyocyte generated from an iPS or iMS cell from an individual comprising a G1801A mutation that results in a G601S substitution in the encoded HERG polypeptide; 29) a cardiomyocyte generated from an iPS or iMS cell from an individual comprising a G1852A mutation that results in a D609N substitution in the encoded HERG polypeptide; 30) a cardiomyocyte generated from an iPS or iMS cell from an individual comprising a T1831C mutation that results in a Y611H substitution in the encoded HERG polypeptide; 31) a cardiomyocyte generated from an iPS or iMS cell from an individual comprising a mutation that results in a substitution in the encoded HERG polypeptide; 32) a cardiomyocyte generated from an iPS or iMS cell from an individual comprising a G1834T mutation that results in a V612L substitution in the encoded HERG polypeptide; 33) a cardiomyocyte generated from an iPS or iMS cell from an individual comprising a C1838T mutation that results in a T613M substitution in the encoded HERG polypeptide; 34) a cardiomyocyte generated from an iPS or iMS cell from an individual comprising a C1841T mutation that results in a A614V substitution in the encoded HERG polypeptide; 35) a cardiomyocyte generated from an iPS or iMS cell from an individual comprising a C1843G mutation that results in an L615V substitution in the encoded HERG polypeptide; 36) a cardiomyocyte generated from an iPS or iMS cell from an individual comprising a G1876A mutation that results in a G626S substitution in the encoded HERG polypeptide; 37) a cardiomyocyte generated from an iPS or iMS cell from an individual comprising a C1881G mutation that results in an F627L substitution in the encoded HERG polypeptide; 38) a cardiomyocyte generated from an iPS or iMS cell from an individual comprising a G1882A mutation that results in a G628S substitution in the encoded HERG polypeptide; 39) a cardiomyocyte generated from an iPS or iMS cell from an individual comprising a A1885G mutation that results in an N629D substitution in the encoded HERG polypeptide; 40) a cardiomyocyte generated from an iPS or iMS cell from an individual comprising a C1887A mutation that results in an N629K substitution in the encoded HERG polypeptide; 41) a cardiomyocyte generated from an iPS or iMS cell from an individual comprising a G1888C mutation that results in a V630L substitution in the encoded HERG polypeptide; 42) a cardiomyocyte generated from an iPS or iMS cell from an individual comprising a T1889C mutation that results in a V630A substitution in the encoded HERG polypeptide; 43) a cardiomyocyte generated from an iPS or iMS cell from an individual comprising a C1894T mutation that results in a P632S substitution in the encoded HERG polypeptide; 44) a cardiomyocyte generated from an iPS or iMS cell from an individual comprising an A1898G mutation that results in an N633S substitution in the encoded HERG polypeptide; 45) a cardiomyocyte generated from an iPS or iMS cell from an individual comprising a C2254T mutation that results in an R752W substitution in the encoded HERG polypeptide; 46) a cardiomyocyte generated from an iPS or iMS cell from an individual comprising a T2414C mutation that results in an F805S substitution in the encoded HERG polypeptide; 47) a cardiomyocyte generated from an iPS or iMS cell from an individual comprising a C2453T mutation that results in an S818L substitution in the encoded HERG polypeptide; 48) a cardiomyocyte generated from an iPS or iMS cell from an individual comprising a G2464A mutation that results in a V822M substitution in the encoded HERG polypeptide; 49) a cardiomyocyte generated from an iPS or iMS cell from an individual comprising a C2467T mutation that results in an R823W substitution in the encoded HERG polypeptide; 50) a cardiomyocyte generated from an iPS or iMS cell from an individual comprising an A2582T mutation that results in an N861I substitution in the encoded HERG polypeptide; 51) a cardiomyocyte generated from an iPS or iMS cell from an individual comprising a C2750T mutation that results in a P917L substitution in the encoded HERG polypeptide; and 52) a cardiomyocyte generated from an iPS or iMS cell from an individual comprising a C2764T mutation that results in an R922W substitution in the encoded HERG polypeptide; where the amino acid numbering based on a known HERG amino acid sequence, e.g., the amino acid sequence depicted in FIG. 2A. Such cardiomyocytes can serve as controls for abnormal cardiomyocytes, e.g., cardiomyocytes that exhibit one or more abnormalities associated with LQTS. In some embodiments, the panel further comprises at least one cardiomyocyte that comprises a wild-type KCNH2 gene encoding wild-type HERG.

In some embodiments, a subject cardiomyocyte panel includes: 1) at least a first cardiomyocyte comprising an LQTS-associated mutation in KCNQ1 encoding KVLQT1; 2) at least a second cardiomyocyte comprising a wild-type KVLQT1-encoding gene, e.g., a KVLQT1-encoding KCNQ1 gene that does not include an LQTS-associated mutation. For example, in some embodiments, a subject cardiomyocyte panel includes a cardiomyocyte comprising an LQTS-associated mutation in KVLQT1 depicted in Table 2 of U.S. Pat. No. 6,787,309 (or Table 1, above).

For example, in some embodiments, a subject cardiomyocyte panel includes a cardiomyocyte comprising an LQTS-associated mutation in a KCNQ1 gene encoding KVLQT1, e.g., the panel includes one or more of: 1) a cardiomyocyte generated from an iPS or iMS cell from an individual comprising a T470G mutation that results in an F157C substitution in the encoded KVLQT1 polypeptide; 2) a cardiomyocyte generated from an iPS or iMS cell from an individual comprising a mutation that results in an A177P substitution in the encoded KVLQT1 polypeptide; 3) a cardiomyocyte generated from an iPS or iMS cell from an individual comprising a mutation that results in a T311I substitution in the encoded KVLQT1 polypeptide; 4) a cardiomyocyte generated from an iPS or iMS cell from an individual comprising a mutation that results in an L272F substitution in the encoded KVLQT1 polypeptide; 5) a cardiomyocyte generated from an iPS or iMS cell from an individual comprising a mutation that results in an R243c substitution in the encoded KVLQT1 polypeptide; 6) a cardiomyocyte generated from an iPS or iMS cell from an individual comprising a mutation that results in a W248R substitution in the encoded KVLQT1 polypeptide; 7) a cardiomyocyte generated from an iPS or iMS cell from an individual comprising a mutation that results in an E261K substitution in the encoded KVLQT1 polypeptide; 8) a cardiomyocyte generated from an iPS or iMS cell from an individual comprising a mutation that results in an E160K substitution in the encoded KVLQT1 polypeptide; 9) a cardiomyocyte generated from an iPS or iMS cell from an individual comprising a mutation that results in a G168R substitution in the encoded KVLQT1 polypeptide; 10) a cardiomyocyte generated from an iPS or iMS cell from an individual comprising a mutation that results in an R174c substitution in the encoded KVLQT1 polypeptide; 11) a cardiomyocyte generated from an iPS or iMS cell from an individual comprising a mutation that results in an A178P substitution in the encoded KVLQT1 polypeptide; 12) a cardiomyocyte generated from an iPS or iMS cell from an individual comprising a mutation that results in an L272F substitution in the encoded KVLQT1 polypeptide; 13) a cardiomyocyte generated from an iPS or iMS cell from an individual comprising a mutation that results in a Y184S substitution in the encoded KVLQT1 polypeptide; 14) a cardiomyocyte generated from an iPS or iMS cell from an individual comprising a mutation that results in an R190Q substitution in the encoded KVLQT1 polypeptide; 15) a cardiomyocyte generated from an iPS or iMS cell from an individual comprising a mutation that results in an A194P substitution in the encoded KVLQT1 polypeptide; 16) a cardiomyocyte generated from an iPS or iMS cell from an individual comprising a mutation that results in an S225L substitution in the encoded KVLQT1 polypeptide; 17) a cardiomyocyte generated from an iPS or iMS cell from an individual comprising a mutation that results in an R243c substitution in the encoded KVLQT1 polypeptide; 18) a cardiomyocyte generated from an iPS or iMS cell from an individual comprising a mutation that results in a W248R substitution in the encoded KVLQT1 polypeptide; 19) a cardiomyocyte generated from an iPS or iMS cell from an individual comprising a mutation that results in an L250H substitution in the encoded KVLQT1 polypeptide; 20) a cardiomyocyte generated from an iPS or iMS cell from an individual comprising a mutation that results in an L266P substitution in the encoded KVLQT1 polypeptide; 21) a cardiomyocyte generated from an iPS or iMS cell from an individual comprising a mutation that results in a G269S substitution in the encoded KVLQT1 polypeptide; 22) a cardiomyocyte generated from an iPS or iMS cell from an individual comprising a mutation that results in an L273F substitution in the encoded KVLQT1 polypeptide; 23) a cardiomyocyte generated from an iPS or iMS cell from an individual comprising a mutation that results in a Y281C substitution in the encoded KVLQT1 polypeptide; 24) a cardiomyocyte generated from an iPS or iMS cell from an individual comprising a mutation that results in an A300T substitution in the encoded KVLQT1 polypeptide; 25) a cardiomyocyte generated from an iPS or iMS cell from an individual comprising a mutation that results in a G306R substitution in the encoded KVLQT1 polypeptide; 26) a cardiomyocyte generated from an iPS or iMS cell from an individual comprising a mutation that results in an L272F substitution in the encoded KVLQT1 polypeptide; 27) a cardiomyocyte generated from an iPS or iMS cell from an individual comprising a mutation that results in a V310I substitution in the encoded KVLQT1 polypeptide; 28) a cardiomyocyte generated from an iPS or iMS cell from an individual comprising a mutation that results in an L272F substitution in the encoded KVLQT1 polypeptide; 29) a cardiomyocyte generated from an iPS or iMS cell from an individual comprising a mutation that results in a P320A substitution in the encoded KVLQT1 polypeptide; 30) a cardiomyocyte generated from an iPS or iMS cell from an individual comprising a mutation that results in an L342F substitution in the encoded KVLQT1 polypeptide; 31) a cardiomyocyte generated from an iPS or iMS cell from an individual comprising a mutation that results in a G345E substitution in the encoded KVLQT1 polypeptide; 32) a cardiomyocyte generated from an iPS or iMS cell from an individual comprising a mutation that results in an S373P substitution in the encoded KVLQT1 polypeptide; 33) a cardiomyocyte generated from an iPS or iMS cell from an individual comprising a mutation that results in an A525T substitution in the encoded KVLQT1 polypeptide; 34) a cardiomyocyte generated from an iPS or iMS cell from an individual comprising a mutation that results in an R555c substitution in the encoded KVLQT1 polypeptide; and 35) a cardiomyocyte generated from an iPS or iMS cell from an individual comprising a mutation that results in a T587M substitution in the encoded KVLQT1 polypeptide. In some embodiments, the panel further comprises at least one cardiomyocyte that comprises a wild-type KCNQ1 gene encoding wild-type KVLQT1.

In some embodiments, a subject cardiomyocyte panel includes: 1) at least a first cardiomyocyte comprising an LQTS-associated mutation in SCN5A (encoding Nav1.5); 2) at least a second cardiomyocyte comprising a wild-type SCN5A gene, e.g., a SCN5A gene that does not include an LQTS-associated mutation. For example, in some embodiments, a subject cardiomyocyte panel includes a cardiomyocyte comprising an LQTS-associated mutation in SCN5A depicted in Table 3, above.

For example, in some embodiments, a subject cardiomyocyte panel includes a cardiomyocyte comprising an LQTS-associated mutation in a SCN5A gene encoding Nav1.5, e.g., the panel includes one or more of: 1) a cardiomyocyte generated from an iPS or iMS cell from an individual comprising a mutation that results in an E1784K substitution in the encoded Nav1.5 polypeptide; 2) a cardiomyocyte generated from an iPS or iMS cell from an individual comprising a mutation that results in an L1825P substitution in the encoded Nav1.5 polypeptide; 3) a cardiomyocyte generated from an iPS or iMS cell from an individual comprising a mutation that results in an R1193Q substitution in the encoded Nav1.5 polypeptide; 4) a cardiomyocyte generated from an iPS or iMS cell from an individual comprising a mutation that results in an N1325S substitution in the encoded Nav1.5 polypeptide; 5) a cardiomyocyte generated from an iPS or iMS cell from an individual comprising a mutation that results in a D1114N substitution in the encoded Nav1.5 polypeptide; 6) a cardiomyocyte generated from an iPS or iMS cell from an individual comprising a mutation that results in a T1304M substitution in the encoded Nav1.5 polypeptide; 7) a cardiomyocyte generated from an iPS or iMS cell from an individual comprising a mutation that results in an L1501V substitution in the encoded Nav1.5 polypeptide; 8) a cardiomyocyte generated from an iPS or iMS cell from an individual comprising a mutation that results in an R1623Q substitution in the encoded Nav1.5 polypeptide; 9) a cardiomyocyte generated from an iPS or iMS cell from an individual comprising a mutation that results in an R1623L substitution in the encoded Nav1.5 polypeptide; 10) a cardiomyocyte generated from an iPS or iMS cell from an individual comprising a mutation that results in a T1645M substitution in the encoded Nav1.5 polypeptide; 11) a cardiomyocyte generated from an iPS or iMS cell from an individual comprising a mutation that results in an S1787N substitution in the encoded Nav10.5 polypeptide; and 11) a cardiomyocyte generated from an iPS or iMS cell from an individual comprising a mutation that results in a D1790G substitution in the encoded Nav1.5 polypeptide. In some embodiments, the panel further comprises at least one cardiomyocyte that comprises wild-type SCN5A gene encoding wild-type Nav1.5.

In some embodiments, a subject cardiomyocyte panel includes: 1) at least a first cardiomyocyte comprising an LQTS-associated mutation in KCNE1; 2) at least a second cardiomyocyte comprising a wild-type KCNE1 gene, e.g., a KCNE1 gene that does not include an LQTS-associated mutation. For example, in some embodiments, a subject cardiomyocyte panel includes a cardiomyocyte comprising an LQTS-associated mutation in KCNE1 depicted in Table 5 of U.S. Pat. No. 6,787,309.

In some embodiments, a subject cardiomyocyte panel includes a cardiomyocyte comprising an LQTS-associated mutation in a KCNE1 gene encoding MinK, e.g., the panel includes one or more of: 1) a cardiomyocyte generated from an iPS or iMS cell from an individual comprising a mutation that results in an A8V substitution in the encoded MinK polypeptide; 2) a cardiomyocyte generated from an iPS or iMS cell from an individual comprising a mutation that results in an R98W substitution in the encoded MinK polypeptide; 3) a cardiomyocyte generated from an iPS or iMS cell from an individual comprising a mutation that results in a T7I substitution in the encoded MinK polypeptide; 4) a cardiomyocyte generated from an iPS or iMS cell from an individual comprising a mutation that results in a V47F substitution in the encoded MinK polypeptide; 5) a cardiomyocyte generated from an iPS or iMS cell from an individual comprising a mutation that results in an L51H substitution in the encoded MinK polypeptide; 6) a cardiomyocyte generated from an iPS or iMS cell from an individual comprising a mutation that results in an S74L substitution in the encoded MinK polypeptide; 7) a cardiomyocyte generated from an iPS or iMS cell from an individual comprising a mutation that results in a D76N substitution in the encoded MinK polypeptide; 8) a cardiomyocyte generated from an iPS or iMS cell from an individual comprising a mutation that results in a W87R substitution in the encoded MinK polypeptide; 9) a cardiomyocyte generated from an iPS or iMS cell from an individual comprising a mutation that results in an R98W substitution in the encoded MinK polypeptide; and a cardiomyocyte generated from an iPS or iMS cell from an individual comprising a mutation that results in a P127T substitution in the encoded MinK polypeptide. In some embodiments, the panel further comprises at least one cardiomyocyte that comprises a wild-type KCNE1 gene encoding wild-type MinK.

In some embodiments, a subject cardiomyocyte panel includes: 1) at least a first cardiomyocyte comprising an LQTS-associated mutation in KCNE2; 2) at least a second cardiomyocyte comprising a wild-type KCNE2 gene, e.g., a KCNE2 gene that does not include an LQTS-associated mutation. For example, in some embodiments, a subject cardiomyocyte panel includes a cardiomyocyte comprising an LQTS-associated mutation in KCNE2 depicted in Table 6 of U.S. Pat. No. 6,787,309.

In some embodiments, a subject cardiomyocyte panel includes a cardiomyocyte comprising an LQTS-associated mutation in a KCNE2 gene encoding miRP1, e.g., the panel includes one or more of: 1) a cardiomyocyte generated from an iPS or iMS cell from an individual comprising a mutation that results in a T8A substitution in the encoded miRP1 polypeptide; 2) a cardiomyocyte generated from an iPS or iMS cell from an individual comprising a mutation that results in a Q9E substitution in the encoded miRP1 polypeptide; 3) a cardiomyocyte generated from an iPS or iMS cell from an individual comprising a mutation that results in an M54T substitution in the encoded miRP1 polypeptide; 4) a cardiomyocyte generated from an iPS or iMS cell from an individual comprising a mutation that results in an 157T substitution in the encoded miRP1 polypeptide; and 5) a cardiomyocyte generated from an iPS or iMS cell from an individual comprising a mutation that results in an A116V substitution in the encoded miRP1 polypeptide. In some embodiments, the panel further comprises at least one cardiomyocyte that comprises a wild-type KCNE2 gene encoding wild-type miRP1.

In some embodiments, a subject cardiomyocyte panel includes two or more cardiomyocytes, each of which includes a different mutation in a different LQTS-associated gene. For example, in some embodiments, a subject cardiomyocyte panel includes: 1) a first cardiomyocyte comprising a first mutation in a first LQTS-associated gene; and 2) at least a second cardiomyocyte comprising a first mutation in a second LQTS-associated gene.

As a non-limiting example, in some embodiments, a subject cardiomyocyte panel includes: 1) a first cardiomyocyte comprising a first mutation in a KCNQ1 gene encoding KVLQT1, where the first mutation results in an A177P substitution, a T311I substitution, an L272F substitution, an R243c substitution, a W248R substitution, or an E261K substitution in KVLQT1, or results in a KVLQT1 mutation as depicted in Table 1, above; 2) a second cardiomyocyte comprising a first mutation in a KCNH2 gene encoding HERG, where the first mutation results in an F291 substitution, an N33T substitution, a G53R substitution, an R56Q substitution, a C66G substitution, an H70R substitution, an A78P substitution, an L86R substitution, or an A490T substitution in HERG, or results in a HERG mutation as depicted in Table 2, above; and 3) a third cardiomyocyte comprising a first mutation in an SCN5A gene encoding Nav1.5, where the first mutation results in an R1193Q substitution, an N1325S substitution, an R1644H substitution, a D1114N substitution, an L1501V substitution, deletion of F1617, an R1623L substitution, or an S1787N substitution in Nav1.5, or results in a Nav1.5 mutation as depicted in Table 3, above. In some embodiments, the panel further includes at least one cardiomyocyte that does not comprise any mutations in any LQTS-associated gene.

### Assessing Effect of a Drug on the QT Interval

The effect of a drug on the QT interval in a cardiomyocyte in a subject cell panel can be determined using any of a variety of methods, a number of which are well known in the art.

Parameters associated with LQTS (e.g., QT interval) that can be assessed include, e.g., measurement of electrical activity of the individual cells. Electrical activity can be measured using well-known and established methods such as extracellular recording; intracellular recording (e.g., patch clamping); and use of voltage-sensitive dyes. The measurements can be carried out using commercial devices, using either whole cell or outside-out patching using both single cell assays as well as automated platforms (see, e.g., Hamill, O P et al Pflugers Arch 1981; 391:85-100, Priest BT and McManus OB Current Pharmaceutical Design 2007; 13:2325-2337). The assay can be optimized using various concentrations of a selective HERG channel blocker (e.g., E4031) and a selective HERG channel activator (e.g., RPR260243), or other substances known to affect ion channels, such as 4-aminopyridine, zatebradine, BaCl₂, lacipidine, benzothiazepine, veratridine and HMR1556.

Suitable voltage-sensitive dyes include, but are not limited to, merocyanine-oxazolone dyes (e.g., NK2367); merocyanine-rhodanine dyes (e.g., NK2495, NK2761, NK2776, NK3224, and NK3225); oxonol dyes (e.g., RH155, RH479, RH482, RH1691, RH1692, and RH1838); styryl dyes (e.g., RH237, RH414, RH421, RH437, RH461, RH795, JPW1063, JPW3028, di-4-ANEPPS, di-9-ANEPPS, di-2-ANEPEQ, di-12-ANEPEQ, di-8-ANEPPQ, di-8-ANEPPS, di-8-ANEPEQ, and di-12-ANEPPQ); and the like. RH237 is N-(4-sulfobutyl)-4(6-(4-(dibutylamino)phenyl)hexatrienyl)pyridinium; inner salt. The ANEP (amino naphthyl ethenyl pyridinium) dyes are suitable for use, where the ANEP dyes include 4-{2-[6-(dibutylamino)-2-naphthalenyl]-ethenyl}-1-(3-sulfopropyl)pyridinium or di-8-butyl-amino-naphthyl-ethylene-pyridinium-propyl-sulfonate (di-8-ANEPPS); di-4-ANEPPS; di-9-ANEPPS; di-2-ANEPEQ; di-12-ANEPEQ; di-8-ANEPPQ; di-8-ANEPEQ; and di-12-ANEPPQ.

Other parameters include changes in intracellular concentration of an ion, e.g., potassium, sodium, or calcium. Changes in the intracellular concentration of an ion such as potassium, sodium, or calcium can be readily detected using a dye that is sensitive to changes in the intracellular concentration of an ion. Various fluorescent dyes that are sensitive to ions such as sodium, potassium, or calcium can be used. For example, to detect changes in intracellular calcium ion concentration, a dye such as arsenazo III, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Aequorin, dextran conjugates or any other derivatives of any of these dyes. To detect changes in intracellular potassium ion concentration, dyes such as potassium-binding benzofuran isophthalate acetoxymethyl ester (PBFI-AM) can be used. To detect changes in intracellular sodium ion concentration, Sodium Green tetraacetate-acetoxy methyl ester, sodium-binding benzofuran isophthalate (SBFI), Sodium Green, CoroNa Green, and the like, can be used. Fluorescence assays can be performed in multi-well plates using plate readers, e.g., FLIPR assay (Fluorescence Image Plate Reader; available from Molecular Devices), e.g. using fluorescent dye indicators.

### Methods of Making a Cardiomyocyte Panel

The present disclosure further provides methods of making a subject cardiomyocyte panel. The methods generally involve: a) generating iPS or iMS cell cells from a plurality of individuals; and b) inducing the iPS cells or iMS cells to differentiate into cardiomyocytes in vitro. The induced cardiomyocytes can be assembled into a panel. Thus, in some embodiments, a subject method of making a cardiomyocyte panel involves a) generating iPS cells or iMS cells from a plurality of individuals; b) inducing the iPS cells or iMS cells to differentiate into cardiomyocytes in vitro; and c) assembling the cardiomyocytes into a panel. Depending on the use to which the panel will be employed, induced cardiomyocytes may be chosen for inclusion in the panel based on a particular mutation of interest, the type of LQTS with which the particular mutation is associated, based on a particular population or sub-population of interest, etc.

### Computer Systems

The present disclosure further provides a computer-readable medium comprising a database that includes information regarding cells in a subject cell panel. The database can comprise data elements and annotations correlating to a subject cell panel. A “data element” represents a property of a cell in a subject cell panel, which can include, e.g., information regarding a mutation in an LQTS-associated gene. Annotations can include information relating to, e.g., 1) the individual from whom the cardiomyocyte originated; and 2) information regarding the effect, if any, of a given drug on the function of an ion channel in a cardiomyocyte in a subject cell panel. Information regarding the individual can include, e.g., sex, age, national origin, race, geographic origin, disease state(s), and the like.

The present disclosure further provides a computer program product that includes a computer readable storage medium comprising a database that includes information regarding cells in a subject cell panel. The computer program product can provide information relating a cell in a subject cell panel to an effect of a drug. Furthermore, a subject computer program product can be used to generate a report, as described below, where the likelihood that a patient will experience LQTS in response to a given drug can be included in such a report.

### Utility

A subject cell, or panel of cells, can be used in a variety of applications. For example, a subject cell or cell panel can be used to assess whether a given compound induces LQTS or has the potential to induce LQTS. A subject cell or panel of cells can be used to optimize a drug treatment for an individual. A subject cell or cell panel can be used to screen test compounds to identify agents that reduce the QT interval and/or ameliorate LQTS. A subject cell or cell panel can be used to identify individuals who are at risk of developing LQTS.

Screening agents for potential to induce LQTS

The present disclosure provides methods for determining whether a given compound has the potential to induce LQTS in an individual (e.g., in an individual who is at higher risk than the general population of developing LQTS due to an LQTS-associated mutation; in a “normal” individual, e.g., an individual who has never experienced an LQTS episode and who does not have any known LQTS-associated mutation).

The methods generally involve contacting a test compound with a subject cardiomyocyte or cells in a subject cardiomyocyte panel, such that the test compound comes into contact with the cardiomyocyte or the cardiomyocytes of the panel, other than a cardiomyocyte(s) that serves as a “compound minus” control; and determining the effect, if any, of the test compound on inducing LQTS in the cardiomyocytes and/or on the function of a potassium, sodium, or calcium ion channel in the cardiomyocyte, e.g., determining the effect, if any, of the compound on the QT interval in the cardiomyocyte.

Compounds to be tested include known compounds; compounds that are investigational new drugs; compounds that are being developed for clinical use; and the like. The compound being tested can be present in a concentration of from about 1 pM to about 100 mM. For example, the cells are present in a liquid medium, and the test compound is added to the liquid medium at a final concentration of from about 1 pM to about 100 mM, e.g., from about 1 pM to about 10 pM, from about 10 pM to about 100 pM, from about 100 pM to about 1 nM, from about 1 nM to about 100 nM, from about 100 nM to about 1 μM, from about 1 μM to about 100 μM, from about 100 μM to about 1 mM, or from about 1 mM to about 100 mM. In some embodiments, the effect of two or more different concentrations of the test compound will be determined. For example, in some embodiments, the effect of the compound at 1 pM, 100 pM, 1 nM, 100 nM, 1 μM, 100 μM, 1 mM, and 100 mM is tested. The effect of the test compound on the QT interval can be compared to the QT interval of a cardiomyocyte not contacted with the test compound.

A test compound that lengthens the QT interval by from about 5% to about 10%, from about 10% to about 15%, from about 15% to about 20%, from about 20% to about 25%, from about 25% to about 30%, from about 30% to about 35%, or more than 35%, can be considered as having the potential to induce LQTS in an individual.

In some embodiments, a subject method of determining whether a compound has the potential to induce LQTS in an individual involves: a) contacting the compound with a cardiomyocyte in vitro, where the cardiomyocyte is generated by inducing an induced pluripotent stem cell obtained from a somatic cell (or other suitable cell, as noted above) from an individual to differentiate into a cardiomyocyte in vitro, where the cardiomyocyte does not comprise a mutation in a polypeptide that controls the QT interval, and where the cardiomyocyte exhibits a normal QT interval, e.g., the cardiomyocyte is a normal cardiomyocyte; and b) determining the effect, if any, of the compound on the QT interval in the cardiomyocyte, compared to the QT interval in the cardiomyocyte in the absence of the compound, wherein an increase in the QT interval in the presence of the compound indicates that the compound can induce LQTS in a susceptible individual.

In some embodiments, a subject method of determining whether a compound has the potential to induce LQTS in an individual involves: a) contacting the compound with a subject cardiomyocyte panel in vitro, where the cardiomyocyte panel comprises “normal” cardiomyocytes (e.g., the cardiomyocytes do not comprise a mutation in a polypeptide that controls the QT interval, and the cardiomyocytes exhibit a normal QT interval under normal physiological conditions); and b) determining the effect, if any, of the compound on the QT interval in the cardiomyocytes in the panel, compared to the QT interval in the cardiomyocytes in the absence of the compound, wherein an increase in the QT interval in the presence of the compound indicates that the compound has the potential to induce LQTS in an individual.

In some embodiments, a subject method of determining whether a compound has the potential to induce LQTS involves: a) contacting at least first and second pluralities of cardiomyocytes with a test compound in vitro, wherein said at least first and second pluralities of cardiomyocytes were generated by differentiating cells, other than embryonic stem cells, from at least two individuals into cardiomyocytes in vitro; and b) determining the effect, if any, of the test compound on the QT interval in the first and second pluralities of cardiomyocytes, compared to the QT interval in the cardiomyocytes in the absence of the compound, wherein an increase in the QT interval in one or more of the pluralities of cardiomyocytes in the presence of the test compound indicates that the test compound has the potential to induce LQTS. In some embodiments, the cells used to generate the cardiomyocytes are iPS cells or iMS cells. In some embodiments, the at least two individuals have not experienced a LQTS episode and who do not have any known LQTS-associated mutations; for example, in some embodiments, the derived cardiomyocytes have a normal QT interval in the absence of the test compound. The at least two individuals can be of various populations or sub-populations. For example, in some embodiments, the at least two individuals comprise are from at least two ethnic groups, and/or are individuals of different human leukocyte antigen haplotypes. In some embodiments, the at least two individuals comprise one or more individuals who have experienced at least one LQTS episode and who have no known LQTS-associated mutations. The at least two individuals can comprise from about 5 individuals to about 1000 individuals, or more than 1000 individuals. The test compound can be any of a variety of compounds, e.g.: an antibiotic, an antihistamine, a heart medication, an anti-fungal agent, an anti-psychotic drug, a blood pressure medication, a cancer chemotherapeutic agent, an anti-viral agent, a drug for treating a neurological disease, an anti-emetic agent, a muscle relaxant, a drug for treating an endocrinological disease, or an immunosuppressive agent.

As discussed above, in some embodiments, a subject cardiomyocyte panel can include normal cardiomyocytes derived from individuals of various populations and/or sub-populations. For example, as discussed above, the cardiomyocyte panel used to test a compound for its potential to induce LQTS in an individual can include cardiomyocytes derived from: 1) individuals of a particular genetic background, e.g., individuals of a particular major histocompatibility complex (MHC) (or human leukocyte antigen; HLA) haplotype, etc.; 2) individuals of a particular race, e.g., Caucasian individuals; African individuals or individuals of African descent; Asian individuals or individuals of Asian descent; native American individuals; African-Americans; Hispanic/Latino individuals; etc.; 3) female individuals; 4) male individuals; 5) individuals having a particular disease state, e.g., individuals with chronic kidney disease such as end-stage renal failure; individuals with known cardiovascular disease; individuals with liver disease; etc.; 6) individuals known to have been exposed chronically to an environmental toxin; 7) individuals who are habitual smokers of tobacco products (e.g., cigarettes); 8) individuals who are considered to be heavy consumers of alcohol; 9) individuals of a particular national or geographic origin; and the like.

In some embodiments, a subject method of determining whether a compound has the potential to induce LQTS in an individual involves: a) contacting the compound with a subject cardiomyocyte panel in vitro, where the cardiomyocyte panel comprises cardiomyocytes derived from individuals who have experienced at least one LQTS episode and who do not have any known LQTS-associated mutation; and b) determining the effect, if any, of the compound on the QT interval in the cardiomyocytes in the panel, compared to the QT interval in the cardiomyocytes in the absence of the compound, wherein an increase in the QT interval in the presence of the compound indicates that the compound has the potential to induce LQTS in an individual.

In some embodiments, a subject method of determining whether a compound has the potential to induce LQTS in an individual involves: a) contacting the compound with a subject cardiomyocyte panel in vitro, where the cardiomyocyte panel comprises: i) “normal” cardiomyocytes (e.g., the cardiomyocytes do not comprise a mutation in a polypeptide that controls the QT interval, and the cardiomyocytes exhibit a normal QT interval under normal physiological conditions); and ii) cardiomyocytes derived from individuals who have experienced at least one LQTS episode and who do not have any known LQTS-associated mutation; and b) determining the effect, if any, of the compound on the QT interval in the cardiomyocytes in the panel, compared to the QT interval in the cardiomyocytes in the absence of the compound, where an increase in the QT interval in the presence of the compound indicates that the compound has the potential to induce LQTS in an individual.

If, using a subject method, a compound is determined to lengthen the QT interval in a normal cardiomyocyte and/or a cardiomyocyte derived from an individual who has no known LQTS-associated mutation and who has experienced at least one LQTS episode, the mechanism by which the compound affects the QT interval can be assessed by comparing the effect of the compound on the QT interval in a normal cardiomyocyte and/or a cardiomyocyte derived from an individual who has no known LQTS-associated mutation and who has experienced at least one LQTS episode, with the characteristics of the QT interval in a cardiomyocyte having an LQTS-associated mutation. Cardiomyocyte having an LQTS-associated mutation include cardiomyocytes comprising an LQTS-associated mutation in a polypeptide selected from KVLQT1, HERG, Nav1.5, Ankyrin B, mink, miRP1, Kir2.1, Cav1.2, caveolin-3, and Navβ4, as described above.

In some embodiments, a test compound that is determined to lengthen the QT interval in a subject assay is further tested for an effect on one or more of: QRS duration and amplitude; local activation time; T wave amplitude; time of maximal slope of T wave; ARI (activation refractory interval).

### Determining the Effect of a Compound on the QT Interval

Methods of determining the effect of the drug on the QT interval include, e.g., measurement of electrical activity of the individual cells. Electrical activity can be measured using well-known and established methods such as extracellular recording (including microelectrode arrays); intracellular recording (e.g., patch clamping); use of voltage-sensitive dyes; use of ion-sensitive dyes; and the like.

The measurements can be carried out using commercial devices, using either whole cell or outside-out patching using both single cell assays as well as automated platforms (see, e.g., Hamill, O P et al Pflugers Arch 1981; 391:85-100, Priest BT and McManus OB Current Pharmaceutical Design 2007; 13:2325-2337). The assay can be optimized using various concentrations of a selective HERG channel blocker (e.g., E4031) and a selective HERG channel activator (e.g., RPR260243), or other substances known to affect ion channels, such as 4-aminopyridine, zatebradine, BaCl₂, lacipidine, benzothiazepine, veratridine and HMR1556. In some embodiments, a known blocker of KVLQT1 (I_{Ks}) is used to optimize the assay; suitable such blockers include, e.g., chromanol 293B (trans-N-[6-Cyano-3,4-dihydro-3-hydroxy-2,2-dimethyl-2H-1-benzopyran-4-yl]-N-methyl-ethanesulfonamide).

In some embodiments, the effect of a test compound on the QT interval is determined using the whole-cell configuration of the patch clamp technique. See, e.g., Rees et al. (1995) J. Gen. Physiol. 106:1151; Liu et al. (1998) Pharmacol. Exp. Ther. 287:877; and Suto et al. (2007) Am. J. Physiol. Heart Circ. Physiol. 292:H1782. Delayed rectifier currents can be measured as peak density of tail current elicited by repolarization to −30 mV following 3-second depolarizing voltage steps from aholding potential of −80 mV. I_{Kr} and I_{Ks} can be measured as the E4031-sensitive and chromanol 293B-sensitive current components, respectively. Such assays can be carried out at physiological temperature (e.g., 35° C.-37° C.).

In some embodiments, a voltage-sensitive dye is used to determine the effect of a test compound on the QT interval. Suitable voltage-sensitive dyes include, but are not limited to, merocyanine-oxazolone dyes (e.g., NK2367); merocyanine-rhodanine dyes (e.g., NK2495, NK2761, NK2776, NK3224, and NK3225); oxonol dyes (e.g., RH155, RH479, RH482, RH1691, RH1692, and RH1838); styryl dyes (e.g., RH237, RH414, RH421, RH437, RH461, RH795, JPW1063, JPW3028, di-4-ANEPPS, di-9-ANEPPS, di-2-ANEPEQ, di-12-ANEPEQ, di-8-ANEPPQ, and di-12-ANEPPQ); and the like.

In other embodiments, microelectrode arrays can be used to determine the effect of a test compound on the QT interval. In this method, extracellular recording brings an electrode close to the cell surface. Microelectrode arrays can be adapted to 96-well or other multi-well plates, and a variety of other formats. See, e.g., Feld et al. (2002) Circulation 105:522-529; and Kehat et al. (2002) Circ Res 9:659.

In some embodiments, the effect of a test compound on the QT interval is determined using an assay that detects changes in intracellular concentration of an ion, e.g., potassium, sodium, or calcium. Changes in the intracellular concentration of an ion such as potassium, sodium, or calcium can be readily detected using a dye that is sensitive to changes in the intracellular concentration of an ion. Various fluorescent dyes that are sensitive to ions such as sodium, potassium, or calcium can be used. For example, to detect changes in intracellular calcium ion concentration, a dye such as arsenazo III, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Aequorin, dextran conjugates or any other derivatives of any of these dyes. To detect changes in intracellular potassium ion concentration, dyes such as potassium-binding benzofuran isophthalate acetoxymethyl ester (PBFI-AM) can be used. To detect changes in intracellular sodium ion concentration, Sodium Green tetraacetate-acetoxy methyl ester, sodium-binding benzofuran isophthalate (SBFI), Sodium Green, CoroNa Green, and the like, can be used. Fluorescence assays can be performed in multi-well plates using plate readers, e.g., FLIPR assay (Fluorescence Image Plate Reader; available from Molecular Devices), e.g. using fluorescent dye indicators.

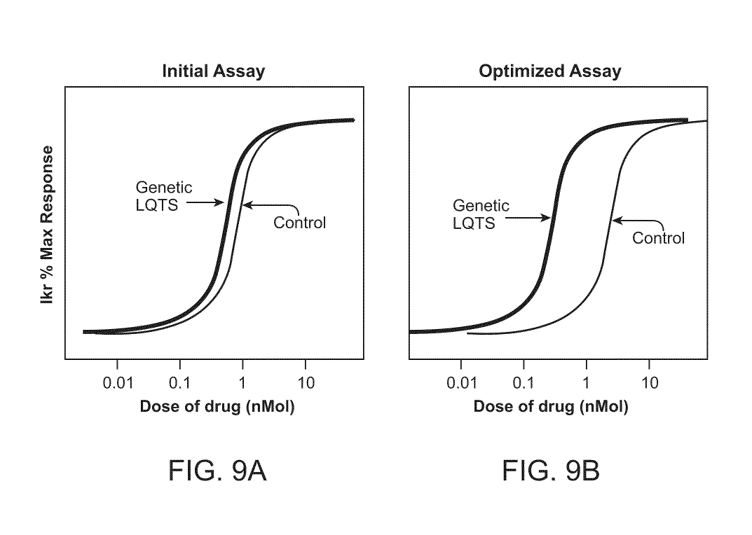

FIGS. 9A and 9B depict an exemplary assay for detecting possible LQTS-inducing activity of a compound. Exemplary data from cardiomyocytes generated from iPS cells or iMS cells in initial versus optimized assays are depicted. FIG. 9A depicts possible responses in an initial assay; and FIG. 9B depicts possible responses in an optimized assay. Dose responses are calibrated with subject cardiomyocytes that comprise LQTS-associated mutations, allowing conditions that provide maximal ability to distinguish a background effect from an effect on the QT interval that might be observed in vivo.

### Treatment Methods

In some embodiments, the present disclosure provides methods for determining a treatment for a patient, the methods generally involving: a) determining whether a given drug being considered for administration to the patient has the potential to induce LQTS in the patient; and b) if the drug being considered has the potential to induce LQTS in the patient, based on the results of (a), prescribing an alternative drug to the drug that has been identified as having the potential to induce LQTS in the patient. Determination of whether a given drug has the potential to induce LQTS in an individual can be performed using a subject method as described above. In some embodiments, an iPS cell or iMS cell is generated using a somatic cell from the individual to be tested, a cardiomyocyte is generated from the iPS cell or iMS cell, and the cardiomyocyte is tested as described above. In some embodiments, one or more alternative drugs are also tested using a subject method.

In some embodiments, the present disclosure provides methods for determining a treatment for a patient, the methods generally involving: a) determining whether a given drug being considered for administration to the patient has the potential to induce LQTS in the patient, where the patient has experienced one or more LQTS episodes in the past and thus is considered at risk of LQTS; and b) if the drug being considered has the potential to induce LQTS in the patient, based on the results of (a), prescribing an alternative drug to the drug that has been identified as having the potential to induce LQTS in the patient. Determination of whether a given drug has the potential to induce LQTS in an individual can be performed using a subject method as described above. In some embodiments, an iPS cell or iMS cell is generated using a somatic cell from the individual to be tested, a cardiomyocyte is generated from the iPS cell or iMS cell, and the cardiomyocyte is tested as described above. In some embodiments, one or more alternative drugs are also tested using a subject method.

As an example, in some embodiments, a subject treatment method involves: a) determining whether an anti-psychotic drug being considered for administration to a patient has the potential to induce LQTS in the patient, where the patient has experienced one or more LQTS episodes in the past and thus is considered at risk of LQTS; and b) if the drug being considered has the potential to induce LQTS in the patient, based on the results of (a), prescribing an alternative drug to the drug that has been identified as having the potential to induce LQTS in the patient. In some embodiments, a cardiomyocyte is generated from a cell (other than an ES cell) obtained from or derived from the patient, and the cardiomyocyte so generated is used to test the anti-psychotic drug being considered for administration to the patient. In some embodiments, a panel of anti-psychotic drugs is tested for an effect on the QT interval in cardiomyocytes generated from a cell (other than an ES cell) obtained from or derived from the patient.

In some embodiments, the present disclosure provides methods for modifying a treatment for a patient, the methods generally involving: a) determining whether a given drug being considered for administered to the patient has the potential to induce LQTS in the patient, where the drug is known to have the potential to induce LQTS in one or more individuals other than the patient; and (b) if the drug being considered has the potential to induce LQTS in the patient, based on the results of (a), prescribing an alternative drug to the drug that has been identified as having the potential to induce LQTS in the patient. In some embodiments, the alternative drug is also tested using a subject method. In some embodiments, a cardiomyocyte is generated from a cell (other than an ES cell) obtained from or derived from the patient, and the cardiomyocyte so generated is used to test the drug being considered for administration to the patient. In some embodiments, a panel of alternative drugs is tested for an effect on the QT interval in cardiomyocytes generated from a cell (other than an ES cell) obtained from or derived from the patient.

As a non-limiting example, in some embodiments a subject method involves: a) determining whether a given cancer chemotherapeutic agent being considered for administered to a cancer patient has the potential to induce LQTS in the patient, where the cancer chemotherapeutic agent is known to have the potential to induce LQTS in one or more individuals other than the patient; and (b) if the cancer chemotherapeutic agent being considered has the potential to induce LQTS in the patient, based on the results of (a), prescribing an alternative cancer chemotherapeutic agent to the cancer chemotherapeutic agent that has been identified as having the potential to induce LQTS in the patient. In some embodiments, the alternative cancer chemotherapeutic agent is also tested using a subject method. In some embodiments, a cardiomyocyte is generated from a cell (other than an ES cell) obtained from or derived from the patient, and the cardiomyocyte so generated is used to test the cancer chemotherapeutic agent being considered for administration to the patient. In some embodiments, a panel of alternative cancer chemotherapeutic agents is tested for an effect on the QT interval in cardiomyocytes generated from a cell (other than an ES cell) obtained from or derived from the patient.

In some embodiments, the present disclosure provides methods for modifying a treatment for a patient, the methods generally involving: a) determining whether a given drug being administered to the patient has the potential to induce LQTS in the patient; and (b) if the drug being considered has the potential to induce LQTS in the patient, based on the results of (a), prescribing an alternative drug to the drug that has been identified as having the potential to induce LQTS in the patient. In some embodiments, the alternative drug is also tested using a subject method.

In some embodiments, the present disclosure provides methods for determining which drug from among two or more drugs being administered to an individual is responsible for inducing LQTS in the individual. The methods generally involve testing each of the drugs being administered to the individual, using a subject method, as described above. In some embodiments, the method further comprising generating a report recommending that a drug that has been identified as inducing LQTS in the individual be either discontinued or be replaced by an alternative drug that does not induce LQTS.

Drugs that can be tested using a subject method include, but are not limited to, 1) antibiotics such as erythromycin, clarithromycin, and other macrolides, fluoroquinolones, sparfloxacin, sulfamethoxazole, trimethoprim, sulfamethoxazole, halofantrine, and pentamidine; 2) antihistamines such as seldane (terfenadine), hismanal (astemizole), azelastine, clemastine, and Benadryl (diphenhydramine); 3) heart medications, such as quinidine, pronestyl, procainamide, disopyramide, dofetilide, sotalol, ibutilide, probucol, bepridil, amiodarone, sotalol, flecamide, moricizine, and tocamide; 4) anti-fungal agents such as ketoconazole, fluconazole, itraconazole; 5) psychotropic drugs (e.g., anti-psychotic drugs) such as amitryptiline, amitryptiline-HCl, amoxapine, desipramine, doxepin, fluvoxamine, imipramine, maprotiline, nortryptiline, fluoxetine, venlafaxine, roxetine, perphenazine, chlorpromazine, clomipramine, fluphenazine, thiothixene, trifluoperazine, phenothiazine derivatives, haloperidol, risperidone, quetiapine, ziprasedone, sertraline, thioridazine, levomethadyl, mesoridazine, and pimozide; 6) blood pressure medication such as indapamide, nicardipine, moexipril, fludrocortisones, and isradipine; 7) cancer medication such as arsenic trioxide, tamoxifen; 8) anti viral drugs such as foscarnet; 9) drugs for treating neurological diseases, e.g., felbamate; fosphenyloin; and selective serotonin agonists such as naratriptan, sumatriptan and zolmitriptan; 10) anti-emetic drugs such as dolasetron, prochlorperazine, and droperidol; 11) muscle relaxants such as tizanidine; 12) drugs for treating pulmonary diseases, e.g., salmeterol; 13) drugs for treating endocrinological diseases, e.g., octreotide; 14) immunosuppressive medication such as tacrolimus; and 15) medication for gastric stimulation such as cisapride.

### Identifying Individuals Who are Susceptible to or are at Risk of Drug-Induced LQTS

A subject cardiomyocyte panel is also useful for identifying individuals who may be susceptible to drug-induced LQTS. A test cardiomyocyte is generated from an iPS or iMS cell generated from a test individual, and included in the panel. The test cardiomyocyte is contacted with an agent or agents known to induce LQTS. Cardiomyocytes that comprise an LQTS-associated mutation in an LQTS-associated gene serve as positive controls. An increase in the QT interval in a test cardiomyocyte when contacted with an agent known to induce LQTS in an individual indicates that the test individual may experience LQTS if treated with the agent. Such a result may indicate that administration of such an agent is contraindicated in the test individual, or that the dose of the agent, if administered to the test individual, should be lowered, or that the test individual should be monitored closely following administration of the agent.

In some embodiments, a subject method of determining whether an individual is at risk of experiencing LQTS in response to a drug involves: a) contacting the drug with a cardiomyocyte in vitro, wherein said cardiomyocyte is generated by inducing an induced pluripotent stem cell obtained from a somatic cell from the individual to differentiate into a cardiomyocyte in vitro; and b) determining the effect, if any, of the drug on the QT interval in the cardiomyocyte, wherein an increase in the QT interval in the presence of the drug indicates that the individual is at risk of experiencing LQTS in response to the drug.

Individuals who would be suitable for screening using a subject method include, e.g., individuals who are being considered for treatment with an agent that has been known to induce LQTS in one or more individuals; an individual who has had palpitations; an individual who has experienced a loss of consciousness; an individual who has undergone resuscitation.

A subject screening method can provide for the likelihood that an individual will experience LQTS. In some embodiments, a patient's likelihood of experiencing LQTS in response to a given drug is provided in a report. Thus, in some embodiments, a subject method further includes a step of preparing or generating a report that includes information regarding the patient's likelihood of LQTS. For example, a subject method can further include a step of generating or outputting a report providing the results of a subject LQTS likelihood assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a subject likelihood assessment and its results. A subject report includes at least a likelihood assessment, e.g., an indication as to the likelihood that a patient will experience LQTS. A subject report can be completely or partially electronically generated. A subject report can further include one or more of: 1) service provider information; 2) patient data; 3) data regarding mutations in LQTS-associated genes; 4) an interpretive report, which can include various information including: a) indication; b) test data, where test data can include: i) effect of drug on QT interval; and/or ii) presence or absence of an LQTS-associated mutation; 5) drug prescription recommendations; and 6) other features.

Screening Assays for Agents that Reduce the QT Interval and/or Ameliorate LQTS

The present disclosure provides assay methods for identifying an agent that reduces the QT interval and/or ameliorates LQTS. A subject method generally involves contacting a subject cardiomyocyte or a subject cardiomyocyte panel with a test agent; and determining the effect, if any, of the test agent on reducing the QT interval in the cardiomyocyte or in a cardiomyocyte in the panel. In some embodiments, a subject method involves: a) contacting a cardiomyocyte or a subject cardiomyocyte panel with a test agent and a compound known to induce LQTS in a normal cardiomyocyte, wherein the cardiomyocyte being contacted with the test agent and the LQTS-inducing compound is a cardiomyocyte that has no known LQTS-associated mutation and, in the absence of the LQTS-inducing compound, has a normal QT interval; and b) determining the effect, if any, of the test agent on reducing the QT interval in the cardiomyocyte or modifying a parameter associated with ion channel function in the cardiomyocyte.

In some embodiments, a subject method of identifying a candidate agent for treating LQTS involves: a) contacting a test agent in vitro with: i) a cardiomyocyte; and ii) an agent that induces LQTS in the cardiomyocyte, wherein the cardiomyocyte is generated by inducing an induced pluripotent stem cell obtained from a somatic cell from an individual to differentiate into a cardiomyocyte in vitro, wherein the cardiomyocyte is a normal cardiomyocyte; and b) determining the effect, if any, of the test agent on the QT interval, or modifying a parameter associated with ion channel function in the cardiomyocyte, wherein a test agent that decreases the QT interval or modifies a parameter associated with ion channel function is a candidate agent for treating LQTS.

In some embodiments, a subject method of identifying a candidate agent for treating LQTS involves: a) contacting a test agent in vitro with a cardiomyocyte that comprises a mutation in a polypeptide that controls the QT interval, such that the cardiomyocyte has a prolonged QT interval; and b) determining the effect, if any, of the test agent on the QT interval, or modifying a parameter associated with ion channel function in the cardiomyocyte, wherein a test agent that decreases the QT interval or modifies a parameter associated with ion channel function is a candidate agent for treating LQTS. A panel of such cardiomyocytes, each of which comprises a mutation in a polypeptide that controls the QT interval, can be used.

Agents that can induce LQTS that are suitable for inclusion in a subject assay include, but are not limited to: 1) antibiotics such as erythromycin, clarithromycin, and other macrolides, fluoroquinolones, sparfloxacin, sulfamethoxazole, trimethoprim, sulfamethoxazole, halofantrine, and pentamidine; 2) antihistamines such as seldane (terfenadine), hismanal (astemizole), azelastine, clemastine, and Benadryl (diphenhydramine); 3) heart medications, such as quinidine, pronestyl, procainamide, disopyramide, dofetilide, sotalol, ibutilide, probucol, bepridil, amiodarone, sotalol, flecamide, moricizine, and tocamide; 4) anti-fungal agents such as ketoconazole, fluconazole, itraconazole; 5) psychotropic drugs such as amitryptiline, amitryptiline-HCl, amoxapine, desipramine, doxepin, fluvoxamine, imipramine, maprotiline, nortryptiline, fluoxetine, venlafaxine, roxetine, perphenazine, chlorpromazine, clomipramine, fluphenazine, thiothixene, trifluoperazine, phenothiazine derivatives, haloperidol, risperidone, quetiapine, ziprasedone, sertraline, thioridazine, levomethadyl, mesoridazine, and pimozide; 6) blood pressure medication such as indapamide, nicardipine, moexipril, fludrocortisones, and isradipine; 7) cancer medication such as arsenic trioxide, tamoxifen; 8) anti viral drugs such as foscarnet; 9) drugs for treating neurological diseases, e.g., felbamate; fosphenyloin; and selective serotonin agonists such as naratriptan, sumatriptan and zolmitriptan; 10) anti-emetic drugs such as dolasetron, prochlorperazine, and droperidol; 11) muscle relaxants such as tizanidine; 12) drugs for treating pulmonary diseases, e.g., salmeterol; 13) drugs for treating endocrinological diseases, e.g., octreotide; 14) immunosuppressive medication such as tacrolimus; and 15) medication for gastric stimulation such as cisapride.

Test agents of interest include agents that reduce the QT interval by at least about 5%, at least about 10%, at least about 15%, at least about 20%, at least about 25%, at least about 30%, at least about 35%, at least about 40%, at least about 45%, at least about 50%, or more, compared to the QT interval in the presence of the LQTS-inducing compound. Test agents of interest are candidate agents for treating LQTS.

Methods of measuring the QT interval are known in the art, and any known method can be used in a subject assay.

Test agents of interest include agents that reduce an adverse effect of an agent known to induce LQTS in a susceptible individual, where test agents of interest reduce the adverse effect by at least about 5%, at least about 10%, at least about 15%, at least about 20%, at least about 25%, at least about 30%, at least about 35%, at least about 40%, at least about 45%, at least about 50%, or more, compared to the effect in the absence of the test agent.

The terms “candidate agent,” “test agent,” “agent,” “substance,” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Assays of the invention include controls, where suitable controls include a cardiomyocyte comprising an LQTS-associated mutation in the presence of the LQTS-inducing compound, but not the test agent. Generally a plurality of assay mixtures is run in parallel with different agent concentrations to obtain a differential response to the various concentrations. Typically, one of these concentrations serves as a negative control, i.e. at zero concentration or below the level of detection.

The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Typically between 0.1 hour and 1 hour will be sufficient.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Screening Drugs for Potential to Induce LQTS

A cardiomyocyte panel is contacted with a drug in development. The effect of the drug on the QT interval is assessed. The effect of the drug on the QT interval is assessed using a patch clamp technique, an external recording method, a voltage-sensitive dye, or an intracellular ion-sensitive dye.

A drug that induces an increase in the QT interval in a normal cardiomyocyte, or in a cardiomyocyte derived from an individual who has experienced at least one LQTS episode and who has no known LQTS-associated mutation, is considered as having the potential to induce LQTS in an individual.

### Generating iPS Cells

Somatic cells are obtained from: 1) normal individuals, i.e., individuals who have no known LQTS-associated mutation and who have not experienced an LQTS episode; and 2) individuals who have experienced at least one LQTS episode and who have no known LQTS-associated mutations.

The somatic cells are induced to become pluripotent stem cells, thus generating iPS cells. Adult human skin fibroblasts are infected with recombinant VSV-g pseudotyped Moloney-based retroviruses comprising nucleotide sequences encoding Oct3/4, Sox2, Klf4, and c-Myc. See, e.g., Dimos et al. (2008) Science 321(5893):1218-21. In some samples, cells are infected with VSV-g pseudotyped retroviruses comprising Oct3/4, Sox2, and Klf4, but not c-Myc. (In some samples, other methods are used to introduce the transgenes. For example, in some samples, piggyBac (PB) transposition is employed to introduce the transgenes, as described in Woltgen, et al. (2009) Nature doi:10.1038/nature 07863, published online Mar. 1, 2009, and Kaji et al. (2009) Nature doi:10.1038/nature07864, published online Mar. 1, 2009.).

Approximately 30,000 fibroblasts are transduced twice over 72 hours, cultured for four days in standard fibroblast medium, and then passaged onto a feeder layer of mouse embryonic fibroblasts in an ES cell supportive medium. Alternatively, cells are seeded on Matrigel-coated plates in MEF-conditioned or non-conditioned primate ES cell medium, both supplemented with 4 ng/ml basic fibroblast growth factor (bFGF). See, e.g., Takahashi et al., (2007) Cell 131 (5):861-72.

In still another alternative, the iPS cells are maintained on irradiated mouse embryonic fibroblasts (MEFs) at a density of about 20,000 cells per cm² in multi-well culture plates. The iPS cells are optionally maintained in DMEM/F12 culture medium supplemented with knockout serum replacement (Invitrogen), nonessential amino acids, L-glutamine, penicillin-streptomycin, and 10 ng/mL basic fibroblast growth factor, as described in Lowry et al. (2008) PNAS 105(8):2883-88.

### Inducing an iPS Cell to Undergo Cardiomyogenesis

Cardiomyocytes (CMs) are generated from iPS cells as follows. Before inducing cardiomyogenesis, the iPS cells are passaged onto a lower density of MEFs (about 10,000 to 15,000 cells per cm²) and expanded for 3 to 4 days. iPS cells are detached from 6-well culture plates by incubating with 1 mg/mL dispase solution, and placed in ultralow attachment plates in suspension culture for 4 days. Differentiation medium (80% DMEM/F12, 0.1 mmol/L nonessential amino acids, 1 mmol/L L-glutamine, 0.1 mmol/L β-mercaptoethanol, and 20% FBS that pretested for cardiac differentiation) is used to initiate cardiac differentiation.

Immunostaining using antibodies specific for α-actinin, sarcomeric myosin heavy chain (MHC), and cardiac Troponin I (cTnI) is performed to confirm the presence of CMs.

### Drug Testing

CMs are plated in 96-well plates in appropriate liquid medium. Test compounds are added to the liquid medium at a concentration of from 1 pM to 100 mM. For example, a test compound is added to cells at concentrations of 1 pM, 100 pM, 1 nM, 100 nM, 1 μM, 100 μM, 1 mM, and 100 mM. Control CMs are not contacted with test compound. The effect of the drug on the QT interval is assessed.

### Electrophysiology

For intracellular electrophysiology experiments, sharp glass microelectrodes are fabricated with resistances of 30-100 MΩ when filled with 3 mol/L KCl. Microelectrodes are inserted into the cells; and pipette capacitance is nulled. Intracellular recordings of membrane potential are made using an Axoclamp-2A amplifier in Bridge Mode (Axon Instruments, Foster City, Calif.), and recordings which show a stable maximum diastolic potential (MDP) for at least 5 minutes are included in data analysis. In some experiments, the preparation is subjected to electrical field stimulation at rates from 1 Hz to 3 Hz. Data are digitized at 20 kHz and filtered at 2 kHz. Action potentials (AP) are analyzed using pClamp8.02 (Axon Instruments, Foster City, Calif.) and Origin 6.0 software (Microcal Inc, Northampton, Mass.) to determine AP duration at 50% and 90% of repolarization (APD50 and APD90), AP amplitude (APA), maximum diastolic potential (MDP), and the maximum rate of rise of the AP upstroke (dV/dtmax).

### High Throughput Screen Using Voltage-Sensitive Dye

A high throughput screen is conducted by using a voltage-sensitive dye such as RH237 or an ANEP dye.

Cardiomyocytes are cultured in a multiwell plate, and are exposed to test compounds over a range of concentrations. The voltage sensitive dyes change absorbance (color), indicating the action potential of the cells. The action potential of a cardiac cell would change with LQTS, allowing for rapid detection of LQTS-inducing drugs. Known LQTS drugs, and genetic forms of LQTS, are used to optimize the test for sensitivity and specificity.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A panel of cardiomyocytes, said panel comprising a plurality of cardiomyocytes, wherein said plurality of cardiomyocytes is generated by inducing cells other than embryonic stem cells obtained from a plurality of individuals to differentiate into cardiomyocytes in vitro.

2. The panel of claim 1, wherein the cells used to generate the cardiomyocytes are induced pluripotent stem (iPS) cells or induced multipotent (iMS) cells.

3. The panel of claim 2, wherein the iPS cells or iMS cells are generated from somatic cells obtained from said plurality of individuals.

4. The panel of claim 1, wherein said plurality of individuals comprises individuals who have not experienced a long QT syndrome (LQTS) episode and who do not have any known LQTS-associated mutations.

5. The panel of claim 1, wherein said plurality of individuals comprise individuals from at least two different ethnic groups.

6. The panel of claim 1, wherein said plurality of individuals comprise individuals of different human leukocyte antigen haplotypes.

7. The panel of claim 1, wherein said plurality of individuals comprises individuals who have experienced at least one LQTS episode and who have no known LQTS-associated mutations.

8. The panel of claim 1, wherein the plurality of individuals comprises from about 5 individuals to about 1000 individuals.

9. The panel of claim 1, wherein said plurality of individuals comprises individuals with a known LQTS-associated mutation.

10. The panel of claim 9, wherein the known LQTS-associated mutation is in a polypeptide selected from HERG, KvLQT1, Nav1.5, ankyrin-B, MinK, MiRP1, Kir2.1, Cav1.2, caveolin-3, and Navβ4.

11. The panel of claim 1, wherein said plurality of individuals are post-natal humans.

12. A method of determining whether a compound has the potential to induce long QT syndrome (LQTS) in an individual, the method comprising:
a) contacting a test compound with a plurality of cardiomyocytes in vitro, said plurality of cardiomyocytes is generated by inducing cells other than embryonic stem cells obtained from a plurality of individuals to differentiate into cardiomyocytes in vitro; and
b) determining the effect, if any, of the test compound on the QT interval in the plurality of cardiomyocytes, compared to the QT interval in the cardiomyocytes in the absence of the compound, wherein an increase in the QT interval in the presence of the test compound indicates that the test compound has the potential to induce LQTS in an individual.

13. The method of claim 12, wherein the cells used to generate the cardiomyocytes are an induced pluripotent stem (iPS) cells.

14. The method of claim 12, wherein said plurality of cardiomyocytes comprises cardiomyocytes that are derived from individuals who have not experienced a long QT syndrome (LQTS) episode and who do not have any known LQTS-associated mutations.

15. The method of claim 14, wherein said derived cardiomyocytes have a normal QT interval.

16. The method of claim 12, wherein said plurality of individuals comprise individuals of at least two different ethnic groups.

17. The method of claim 12, wherein said plurality of individuals comprise individuals of different human leukocyte antigen haplotypes.

18. The method of claim 12, wherein said plurality of individuals comprises individuals who have experienced at least one LQTS episode and who have no known LQTS-associated mutations.

19. The method of claim 18, wherein said individuals are post-natal humans.

20. The method of claim 12, wherein the plurality of individuals comprises from about 5 individuals to about 1000 individuals.

21. The method of claim 12, wherein the test compound is an antibiotic, an antihistamine, a heart medication, an anti-fungal agent, an anti-psychotic drug, a blood pressure medication, a cancer chemotherapeutic agent, an anti-viral agent, a drug for treating a neurological disease, an anti-emetic agent, a muscle relaxant, a drug for treating an endocrinological disease, or an immunosuppressive agent.

22. A method of determining whether a compound has the potential to induce long QT syndrome (LQTS), the method comprising:
a) contacting at least first and second pluralities of cardiomyocytes with a test compound in vitro, wherein said at least first and second pluralities of cardiomyocytes were generated by differentiating cells, other than embryonic stem cells, from at least two individuals into cardiomyocytes in vitro; and
b) determining the effect, if any, of the test compound on the QT interval in the first and second pluralities of cardiomyocytes, compared to the QT interval in the cardiomyocytes in the absence of the compound, wherein an increase in the QT interval in one or more of the pluralities of cardiomyocytes in the presence of the test compound indicates that the test compound has the potential to induce LQTS.

23. The method of claim 22, wherein the cells used to generate the cardiomyocytes are induced pluripotent stem (iPS) cells or induced multipotent stem (iMS) cells.

24. The method of claim 22, wherein said at least two individuals have not experienced a long QT syndrome (LQTS) episode and who do not have any known LQTS-associated mutations.

25. The method of claim 24, wherein said derived cardiomyocytes have a normal QT interval in the absence of the test compound.

26. The method of claim 22, wherein said at least two individuals comprise are from at least two ethnic groups.

27. The method of claim 22, wherein said at least two individuals comprise individuals of different human leukocyte antigen haplotypes.

28. The method of claim 22, wherein said at least two individuals comprise one or more individuals who have experienced at least one LQTS episode and who have no known LQTS-associated mutations.

29. The method of claim 22, wherein said at least two individuals are post-natal humans.

30. The method of claim 22, where said at least two individuals comprise from about 5 individuals to about 1000 individuals.

31. The method of claim 22, wherein the test compound is an antibiotic, an antihistamine, a heart medication, an anti-fungal agent, an anti-psychotic drug, a blood pressure medication, a cancer chemotherapeutic agent, an anti-viral agent, a drug for treating a neurological disease, an anti-emetic agent, a muscle relaxant, a drug for treating an endocrinological disease, or an immunosuppressive agent.

32. A method of treating an individual, the method comprising:
a) determining whether a drug being considered for administration to an individual has the potential to induce long QT syndrome (LQTS) in the individual;
b) determining a treatment regimen based on the results of step (a).

33. The method of claim 32, wherein said determining comprises contacting said drug under consideration with a normal cardiomyocyte derived from a cell, other than an ES cell, obtained from an individual who has not experienced a LQTS episode and who does not have any known LQTS-associated mutation, and determining the effect of the drug under consideration on the QT interval in said normal cardiomyocyte.

34. The method of claim 32, wherein said determining comprises contacting said drug under consideration with a cardiomyocyte derived from a cell, other than an ES cell, obtained from said individual, and determining the effect of the drug under consideration on the QT interval in said cardiomyocyte.

35. The method of claim 32, wherein, if said drug under consideration is determined to have the potential to induce LQTS in the individual, then step (b) comprises prescribing an alternative drug.

36. The method of claim 32, further comprising generating a report indicating whether the drug is likely to induce LQTS in the individual.

37. The method of claim 33, wherein said cell from which said cardiomyocyte is generated is an induced pluripotent cell or an induced multipotent cell.

38. The method of claim 34, wherein said cell from which said cardiomyocyte is generated is an induced pluripotent cell or an induced multipotent cell.

39. A method of determining whether an individual is at risk of experiencing drug-induced long QT syndrome (LQTS) in response to a drug, the method comprising:
a) contacting the drug with a cardiomyocyte in vitro, wherein said cardiomyocyte is generated by differentiating an induced pluripotent stem cell or an induced multipotent cell obtained from a somatic cell from the individual into a cardiomyocyte in vitro; and
b) determining the effect, if any, of the drug on the QT interval in the cardiomyocyte, wherein an increase in the QT interval in the presence of the drug indicates that the individual is at risk of experiencing LQTS in response to the drug.

40. The method of claim 39, wherein the drug is an antibiotic, an antihistamine, a heart medication, an anti-fungal agent, a psychotropic drug, a blood pressure medication, a cancer chemotherapeutic agent, an anti-viral agent, a drug for treating a neurological disease, an anti-emetic agent, a muscle relaxant, a drug for treating an endocrinological disease, or an immunosuppressive agent.

41. A method of identifying a candidate agent for treating long QT syndrome (LQTS), the method comprising:
a) contacting a cardiomyocyte in vitro with:
i) a test agent; and
ii) an agent that induces LQTS in the cardiomyocyte; and
b) determining the effect, if any, of the test agent on the QT interval in said cardiomyocyte, wherein said cardiomyocytes were generated by differentiating an induced pluripotent stem cell, obtained from a somatic cell from an individual, into cardiomyocytes in vitro, and wherein a test agent that decreases the QT interval, or reverses the agent-induced increase in the QT interval, is a candidate agent for treating LQTS.

42. The method of claim 41, wherein said cardiomyocyte is a normal cardiomyocyte derived from a cell, other than an ES cell, from an individual who has not experienced a LQTS episode and who does not have any LQTS-associated mutation.

43. The method of claim 41, wherein said LQTS-inducing agent is selected from an antibiotic, and anti-fungal agent, an anti-arrhythmia agent, a psychoactive agent, and a diuretic.


SEQUENCE LISTING (9 sequences)

SEQ ID NO: 1 - protein, 676 aa, Homo sapiens [sequence omitted]

SEQ ID NO: 2 - protein, 676 aa, Homo sapiens [sequence omitted]

SEQ ID NO: 3 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 4 - protein, 2016 aa, Homo sapiens
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPVRRAAVKILVHSLFNMLIMCTILTNCVFMAQHDPPPWTKYVEYTFTAIYTFESLVKILARAFCLHAFTFLRDPWNWLDFSVIIMAYTTEFVDLGNVSALRTFRVLRALKTISVISGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTARESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRHRPQKPAALAAQGQLPSCIATPYSPPPPETEKVPPTRKETQFEEGEQPGQGTPGDPEPVCVPIAVAESDTDDQEEDEENSLGTEEESSKQQESQPVSGWPRGPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 5 - protein, 3957 aa, Homo sapiens
MMNEDAAQKSDSGEKFNGSSQRRKRPKKSDSNASFLRAARAGNLDKVVEYLKGGIDINTCNQNGLNALHLAAKEGHVGLVQELLGRGSSVDSATKKGNTALHIASLAGQAEVVKVLVKEGANINAQSQNGFTPLYMAAQENHIDVVKYLLENGANQSTATEDGFTPLAVALQQGHNQAVAILLENDTKGKVRLPALHIAARKDDTKSAALLLQNDHNADVQSKMMVNRTTESGFTPLHIAAHYGNVNVATLLLNRGAAVDFTARNGITPLHVASKRGNTNMVKLLLDRGGQIDAKTRDGLTPLHCAARSGHDQVVELLLERGAPLLARTKNGLSPLHMAAQGDHVECVKHLLQHKAPVDDVTLDYLTALHVAAHCGHYRVTKLLLDKRANPNARALNGFTPLHIACKKNRIKVMELLVKYGASIQAITESGLTPIHVAAFMGHLNIVLLLLQNGASPDVTNIRGETALHMAARAGQVEVVRCLLRNGALVDARAREEQTPLHIASRLGKTEIVQLLLQHMAHPDAATTNGYTPLHISAREGQVDVASVLLEAGAAHSLATKKGFTPLHVAAKYGSLDVAKLLLQRRAAADSAGKNGLTPLHVAAHYDNQKVALLLLEKGASPHATAKNGYTPLHIAAKKNQMQIASTLLNYGAETNIVTKQGVTPLHLASQEGHTDMVTLLLDKGANIHMSTKSGLTSLHLAAQEDKVNVADILTKHGADQDAHTKLGYTPLIVACHYGNVKMVNFLLKQGANVNAKTKNGYTPLHQAAQQGHTHIINVLLQHGAKPNATTANGNTALAIAKRLGYISVVDTLKVVTEEVTTTTTTITEKHKLNVPETMTEVLDVSDEEGDDTMTGDGGEYLRPEDLKELGDDSLPSSQFLDGMNYLRYSLEGGRSDSLRSFSSDRSHTLSHASYLRDSAVMDDSVVIPSHQVSTLAKEAERNSYRLSWGTENLDNVALSSSPIHSGFLVSFMVDARGGAMRGCRHNGLRIIIPPRKCTAPTRVTCRLVKRHRLATMPPMVEGEGLASRLIEVGPSGAQFLGKLHLPTAPPPLNEGESLVSRILQLGPPGTKFLGPVIVEIPHFAALRGKERELVVLRSENGDSWKEHFCDYTEDELNEILNGMDEVLDSPEDLEKKRICRIITRDFPQYFAVVSRIKQDSNLIGPEGGVLSSTVVPQVQAVFPEGALTKRIRVGLQAQPMHSELVKKILGNKATFSPIVTLEPRRRKFHKPITMTIPVPKASSDVMLNGFGGDAPTLRLLCSITGGTTPAQWEDITGTTPLTFVNECVSFTTNVSARFWLIDCRQIQESVTFASQVYREIICVPYMAKFVVFAKSHDPIEARLRCFCMTDDKVDKTLEQQENFAEVARSRDVEVLEGKPIYVDCFGNLVPLTKSGQHHIFSFFAFKENRLPLFVKVRDTTQEPCGRLSFMKEPKSTRGLVHQAICNLNITLPIYTKESESDQEQEEEIDMTSEKNDETESTETSVLKSHLVNEVPVLASPDLLSEVSEMKQDLIKMTAILTTDVSDKAGSIKVKELVKAAEEEPGEPFEIVERVKEDLEKVNEILRSGTCTRDESSVQSSRSERGLVEEEWVIVSDEEIEEARQKAPLEITEYPCVEVRIDKEIKGKVEKDSTGLVNYLTDDLNTCVPLPKEQLQTVQDKAGKKCEALAVGRSSEKEGKDIPPDETQSTQKQHKPSLGIKKPVRRKLKEKQKQKEEGLQASAEKAELKKGSSEESLGEDPGLAPEPLPTVKATSPLIEETPIGSIKDKVKALQKRVEDEQKGRSKLPIRVKGKEDVPKKTTHRPHPAASPSLKSERHAPGSPSPKTERHSTLSSSAKTERHPPVSPSSKTEKHSPVSPSAKTERHSPASSSSKTEKHSPVSPSTKTERHSPVSSTKTERHPPVSPSGKTDKRPPVSPSGRTEKHPPVSPGRTEKRLPVSPSGRTDKHQPVSTAGKTEKHLPVSPSGKTEKQPPVSPTSKTERIEETMSVRELMKAFQSGQDPSKHKTGLFEHKSAKQKQPQEKGKVRVEKEKGPILTQREAQKTENQTIKRGQRLPVTGTAESKRGVRVSSIGVKKEDAAGGKEKVLSHKIPEPVQSVPEEESHRESEVPKEKMADEQGDMDLQISPDRKTSTDFSEVIKQELEDNDKYQQFRLSEETEKAQLHLDQVLTSPFNTTFPLDYMKDEFLPALSLQSGALDGSSESLKNEGVAGSPCGSLMEGTPQISSEESYKHEGLAETPETSPESLSFSPKKSEEQTGETKESTKTETTTEIRSEKEHPTTKDITGGSEERGATVTEDSETSTESFQKEATLGSPKDTSPKRQDDCTGSCSVALAKETPTGLTEEAACDEGQRTFGSSAHKTQTDSEVQESTATSDETKALPLPEASVKTDTGTESKPQGVIRSPQGLELALPSRDSEVLSAVADDSLAVSHKDSLEASPVLEDNSSHKTPDSLEPSPLKESPCRDSLESSPVEPKMKAGIFPSHFPLPAAVAKTELLTEVASVRSRLLRDPDGSAEDDSLEQTSLMESSGKSPLSPDTPSSEEVSYEVTPKTTDVSTPKPAVIHECAEEDDSENGEKKRFTPEEEMFKMVTKIKMFDELEQEAKQKRDYKKEPKQEESSSSSDPDADCSVDVDEPKHTGSGEDESGVPVLVTSESRKVSSSSESEPELAQLKKGADSGLLPEPVIRVQPPSPLPSSMDSNSSPEEVQFQPVVSKQYTFKMNEDTQEEPGKSEEEKDSESHLAEDRHAVSTEAEDRSYDKLNRDTDQPKICDGHGCEAMSPSSSAAPVSSGLQSPTGDDVDEQPVIYKESLALQGTHEKDTEGEELDVSRAESPQADCPSESFSSSSSLPHCLVSEGKELDEDISATSSIQKTEVTKTDETFENLPKDCPSQDSSITTQTDRFSMDVPVSDLAENDEIYDPQITSPYENVPSQSFFSSEESKTQTDANHTTSFHSSEVYSVTITSPVEDVVVASSSSGTVLSKESNFEGQDIKMESQQESTLWEMQSDSVSSSFEPTMSATTTVVGEQISKVIITKTDVDSDSWSEIREDDEAFEARVKEEEQKIFGLMVDRQSQGTTPDTTPARTPTEEGTPTSEQNPFLFQEGKLFEMTRSGAIDMTKRSYADESFHFFQIGQESREETLSEDVKEGATGADPLPLETSAESLALSESKETVDDEADLLPDDVSEEVEEIPASDAQLNSQMGISASTETPTKEAVSVGTKDLPTVQTGDIPPLSGVKQISCPDSSEPAVQVQLDFSTLTRSVYSDRGDDSPDSSPEEQKSVIEIPTAPMENVPFTESKSKIPVRTMPTSTPAPPSAEYESSVSEDFLSSVDEENKADEAKPKSKLPVKVPLQRVEQQLSDLDTSVQKTVAPQGQDMASIAPDNRSKSESDASSLDSKTKCPVKTRSYTETETESRERAEELELESEEGATRPKILTSRLPVKSRSTTSSCRGGTSPTKESKEHFFDLYRNSIEFFEEISDEASKLVDRLTQSEREQEIVSDDESSSALEVSVIENLPPVETEHSVPEDIFDTRPIWDESIETLIERIPDENGHDHAEDPQDEQERIEERLAYIADHLGFSWTELARELDFTEEQIHQIRIENPNSLQDQSHALLKYWLERDGKHATDTNLVECLTKINRMDIVHLMETNTEPLQERISHSYAEIEQTITLDHSEGFSVLQEELCTAQHKQKEEQAVSKESETCDHPPIVSEEDISVGYSTFQDGVPKTEGDSSATALFPQTHKEQVQQDFSGKMQDLPEESSLEYQQEYFVTTPGTETSETQKAMIVPSSPSKTPEEVSTPAEEEKLYLQTPTSSERGGSPIIQEPEEPSEHREESSPRKTSLVIVESADNQPETCERLDEDAAFEKGDDMPEIPPETVTEEEYIDEHGHTVVKKVTRKIIRRYVSSEGTEKEEIMVQGMPQEPVNIEEGDGYSKVIKRVVLKSDTEQSEDNNE

SEQ ID NO: 6 - protein, 1872 aa, Homo sapiens
MMNEDAAQKSDSGEKFNGSSQRRKRPKKSDSNASFLRAARAGNLDKVVEYLKGGIDINTCNQNGLNALHLAAKEGHVGLVQELLGRGSSVDSATKKGNTALHIASLAGQAEVVKVLVKEGANINAQSQNGFTPLYMAAQENHIDVVKYLLENGANQSTATEDGFTPLAVALQQGHNQAVAILLENDTKGKVRLPALHIAARKDDTKSAALLLQNDHNADVQSKMMVNRTTESGFTPLHIAAHYGNVNVATLLLNRGAAVDFTARNGITPLHVASKRGNTNMVKLLLDRGGQIDAKTRDGLTPLHCAARSGHDQVVELLLERGAPLLARTKNGLSPLHMAAQGDHVECVKHLLQHKAPVDDVTLDYLTALHVAAHCGHYRVTKLLLDKRANPNARALNGFTPLHIACKKNRIKVMELLVKYGASIQAITESGLTPIHVAAFMGHLNIVLLLLQNGASPDVTNIRGETALHMAARAGQVEVVRCLLRNGALVDARAREEQTPLHIASRLGKTEIVQLLLQHMAHPDAATTNGYTPLHISAREGQVDVASVLLEAGAAHSLATKKGFTPLHVAAKYGSLDVAKLLLQRRAAADSAGKNGLTPLHVAAHYDNQKVALLLLEKGASPHATAKNGYTPLHIAAKKNQMQIASTLLNYGAETNIVTKQGVTPLHLASQEGHTDMVTLLLDKGANIHMSTKSGLTSLHLAAQEDKVNVADILTKHGADQDAHTKLGYTPLIVACHYGNVKMVNFLLKQGANVNAKTKNGYTPLHQAAQQGHTHIINVLLQHGAKPNATTANGNTALAIAKRLGYISVVDTLKVVTEEVTTTTTTITEKHKLNVPETMTEVLDVSDEEGDDTMTGDGGEYLRPEDLKELGDDSLPSSQFLDGMNYLRYSLEGGRSDSLRSFSSDRSHTLSHASYLRDSAVMDDSVVIPSHQVSTLAKEAERNSYRLSWGTENLDNVALSSSPIHSGFLVSFMVDARGGAMRGCRHNGLRIIIPPRKCTAPTRVTCRLVKRHRLATMPPMVEGEGLASRLIEVGPSGAQFLGKLHLPTAPPPLNEGESLVSRILQLGPPGTKFLGPVIVEIPHFAALRGKERELVVLRSENGDSWKEHFCDYTEDELNEILNGMDEVLDSPEDLEKKRICRIITRDFPQYFAVVSRIKQDSNLIGPEGGVLSSTVVPQVQAVFPEGALTKRIRVGLQAQPMHSELVKKILGNKATFSPIVTLEPRRRKFHKPITMTIPVPKASSDVMLNGFGGDAPTLRLLCSITGGTTPAQWEDITGTTPLTFVNECVSFTTNVSARFWLIDCRQIQESVTFASQVYREIICVPYMAKFVVFAKSHDPIEARLRCFCMTDDKVDKTLEQQENFAEVARSRDVEVLEGKPIYVDCFGNLVPLTKSGQHHIFSFFAFKENRLPLFVKVRDTTQEPCGRLSFMKEPKSTRGLVHQAICNLNITLPIYTKESESDQEQEEEIDMTSEKNPQDEQERIEERLAYIADHLGFSWTELARELDFTEEQIHQIRIENPNSLQDQSHALLKYWLERDGKHATDTNLVECLTKINRMDIVHLMETNTEPLQERISHSYAEIEQTITLDHSEGFSVLQEELCTAQHKQKEEQAVSKESETCDHPPIVSEEDISVGYSTFQDGVPKTEGDSSATALFPQTHKEQVQQDFSGKMQDLPEESSLEYQQEYFVTTPGTETSETQKAMIVPSSPSKTPEEVSTPAEEEKLYLQTPTSSERGGSPIIQEPEEPSEHREESSPRKTSLVIVESADNQPETCERLDEDAAFEKGDDMPEIPPETVTEEEYIDEHGHTVVKKVTRKIIRRYVSSEGTEKEEIMVQGMPQEPVNIEEGDGYSKVIKRVVLKSDTEQSEDNNE

SEQ ID NO: 7 - protein, 129 aa, Homo sapiens
MILSNTTAVTPFLTKLWQETVQQGGNMSGLARRSPRSSDGKLEALYVLMVLGFFGFFTLGIMLSYIRSKKLEHSNDPFNVYIESDAWQEKDKAYVQARVLESYRSCYVVENHLAIEQPNTHLPETKPSP

SEQ ID NO: 8 - protein, 123 aa, Homo sapiens
MSTLSNFTQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGAAGFKMSP

SEQ ID NO: 9 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI